(19) United States
(12) Patent Application Publication
(10) Pub. No.: US 2026/0347908 A1
(43) Pub. Date: Nov. 5, 2026

(54) METHOD FOR DETERMINING THE NUMBER AND/OR CONCENTRATION OF GERMINABLE CELLS IN A SAMPLE OF BIOLOGICAL ORIGIN

(71) Applicant: Evonik Operations GmbH, Essen (DE)

(72) Inventors: Mehmet SAHIN, Hanau (DE); Michelle DARGATZ, Bielefeld (DE); Emeka Ignatius IGWE, Muenchen (DE); Ingolf REIMANN, Reinheim (DE); Markus WILTAFSKY-MARTIN, Moembris (DE)

(73) Assignee: Evonik Operations GmbH, Essen (DE)

(21) Appl. No.: 19/134,785

(22) PCT Filed: Dec. 12, 2023
(86) PCT No.: PCT/EP2023/085354

§ 371 Date: Jun. 2, 2025

(30) Foreign Application Priority Data
Dec. 15, 2022 (EP) 22213736

(51) Int. Cl. C12Q 1/6806; C12Q 1/6851

(52) U.S. Cl. CPC C12Q 1/6806; C12Q 1/6851

(57) ABSTRACT

The present invention relates to a method for determining the number and/or concentration of germinable cells in a sample of biological origin, wherein the germinable cells are bacterial spores formed within vegetative cells, comprising the steps of a) subjecting at least a part of a sample material of biological origin to a pre-treatment, involving a lysis of vegetative cells, b) photolyzing the pre-treated sample material obtained from step a), c) separating nucleic acids from the photolyzed sample material obtained from step b), and d) subjecting the separated nucleic acids obtained from step c) to a quantitative polymerase chain reaction to obtain the number and/or concentration of germinable cells in the sample material of step a), characterized in that the pre-treatment of step a) comprises the step of treating the sample material with a basic solution.

The present invention relates to a method for determining the number and/or concentration of germinable cells in a sample of biological origin, wherein the germinable cells are bacterial spores formed within vegetative cells, which involves a pre-treatment of the sample material prior to being subjected to a quantitative polymerase chain reaction with a basic solution.

Some food or feed products contain bacteria which are beneficial for the respective living being, specifically for the gut care of the living being. For example, Evonik's products Ecobiol®, GutCare®, and GutPlus® are feed additives which contain a natural fast-growing B. amyloliquefaciens and/or B. subtilis. These products improve the condition of the animal and help producers to solve quality, profitability, and sustainability challenges. The quality of such a product is directly associated with the number of germinable cells contained in that product.

The number of total cells includes both viable and non-viable cells in a particular culture. Viable cells, also referred to as alive cells, are cells that are able of living, germinating, and growing. By comparison, non-viable cells, also referred to as dead cells are the cells that cannot grow to produce colonies in a cell culture. They are not capable of living, germinating, or reproducing. The group of viable cells includes both vegetative and germinable cells. Vegetative cells are normal growing cells. They are metabolically active and functioning and have the ability of sporulating. While the vegetative cell is the active form of bacteria cells, the spore can be thought of as a dormant form of the cell. It allows for survival of adverse conditions, but it does not allow the cell to grow or reproduce. Their effect is exhibited mainly in the gastrointestinal tract. However, vegetative cells are not resistant to harsh environmental conditions and cannot survive without nutrients. Germinable cells are bacterial spores formed within vegetative cells. They have the ability to germinate. Further, they are also resistant to physical and chemical treatments, which allow the persistence and dissemination of the bacterial species. In contrast to vegetative cells, germinable cells can survive without nutrients. Therefore, germinable cells must be differentiated from all other cells, i.e., non-viable and vegetative cells, in any product as well as in any samples of biomass, feed, fecal and digesta samples. This involves the determination of the germinable cell concentration in the samples.

The microbial spore count is one approach for determining the concentration of germinable cells in samples. The concentrations values obtained with the microbial spore count are expressed in CFU (colony-forming units) per gram or milliliter, which equals the concentration of germinable cells present in the sample. The microbial spore count allows for a differentiation between phenotypes (colony morphology). However, it is difficult to differentiate on a strain level or even between close species using this method. Rather, the method is only able of counting the bacteria, that can form colonies under the applied condition. If species or strain identification is required, each colony needs to be exposed to suitable methods such as MALDI-TOF, 16s sequencing or a specific identity PCR.

Another disadvantage of the microbial spore count is its low precision, which is also matrix and organism/cell dependent. The slow speed of the method is a further handicap: the formation of visible and countable colonies takes at least 24 hours or up to 48 hours depending on the organism. Further significant drawbacks of this method are for example that it is time- and work-intensive, and wasteful with respect to many disposable items, such as plastic pipettes and petri dishes. Nevertheless, the microbial spore count became as a type of gold standard for determining the concentration of germinable cells in samples and is still used as a standard for determining the concentration of germinable cells in samples.

The quantitative polymerase chain reaction (qPCR) is a DNA-based method for the quantification of a target gene after extraction of nucleic acids from a sample. This method allows for the differentiation between species, strains, and even genotypes with a difference of few bases.

For example, US 2018/0037936 A1 discloses a method for quantitative monitoring of bacterial endospores in an aqueous environment of a paper or board mill, which involves destroying vegetative bacteria in a first sample by a suitable treatment, e.g., heating, followed by photolysis using, e.g., propidium monoazide (PMA). The heating is most preferably at least 75 °C. Also disclosed is a method further comprising a step of determining the amount of vegetative cells in a second sample and comparing the determined endospore levels from the first sample to the amount of vegetative bacterial cells in the second sample.

US 2011/0318750 A1 discloses a method that allows the detection and quantification of viable bacterial endospores from samples of both low and nigh biomass. A pre-treatment step of heating at 80/85 °C for about 15 minutes is performed to inactivate/kill viable bacterial cells in the sample. The sample is then labelled with PMA for photolysis, nucleic acids are extracted and subjected to qPCR.

The article “PCR-Based Method Using Propidium Monoazide to Distinguish Viable from Nonviable Bacillus subtilis Spores” (Rawsthorne, H. et al., Applied and Environmental Microbiology, 2009, Vol. 75, No. 9, 2936-3939) describes the determination of the number and/or concentration of viable B. subtilis spores from overnight cultures. The vegetative cells were spread into sporulation agar and exposed to aerobic conditions resulting into 95% of sporulated cells. The harvested spores were further exposed to heat (115 °C in oil bath) to mimic thermal inactivation procedures in food-processing technologies (pre-treatment involving lysis of vegetative cells). Suspensions were then treated with PMA (photolysis), DNA was isolated and subjected to quantitative PCR. The PMA-qPCR method was compared to the “gold standard” enumeration by plating method as well as to qPCR without PMA treatment. The identification of viable bacterial spores based on PMA-qPCR is discussed as an alternative to cultural methods in validation of food-processing efficacy.

Depending on the extraction method, qPCR has a high precision. The time-to-result ranges from 3 to 4 hours, which is relatively short, compared to microbial counting. However, the disadvantage of this method is that it counts all target cells, regardless of their viability status. In other words, qPCR is not able to distinguish between dead, vegetative or germinable cells.

Accordingly, there was still a need for a method which allows for the specific and precise determination of the number and/or concentration of germinable cells in samples of biological origin.

It was found that this problem is solved in that the quantitative polymerase chain reaction is preceded by an additional pre-treatment of the sample material. The sample material to be subjected to the pre-treatment contains the group of viable cells, which can induce germination under specific conditions, and the non-viable cells. Hence, said sample material contains the non-viable (dead) cells, and the viable (living or alive) cells, which comprises the vegetative and the germinable cells.

At least a part of the sample material is first subjected to a pre-treatment which involves a lysis of vegetative cells. This step aims at making germinable cells distinguishable from all other cells, i.e., vegetative cells and dead (non-viable) cells. Subsequently, the thus pre-treated sample material is subjected to a photolysis with a photoreactive DNA binding dye. Next, the nucleic acids are separated from the thus obtained photolyzed sample material and then subjected to a qPCR to obtain the number of germinable cells in the original sample material. This procedure has the effect that during the qPCR only the DNA derived from intact cells is amplified.

Object of the present invention is therefore a method for determining the number and/or concentration of germinable cells in a sample of biological origin, wherein the germinable cells are bacterial spores formed within the vegetative cells, comprising the steps of
a) subjecting at least a part of a sample material of biological origin to a pre-treatment, involving a lysis of vegetative cells,
b) photolyzing the pre-treated sample material obtained from step a),
c) separating nucleic acids from the photolyzed sample material obtained from step b), and
d) subjecting the separated nucleic acids obtained from step c) to a quantitative polymerase chain reaction to obtain the number and/or concentration of germinable cells in the sample material of step a),
characterized in that the pre-treatment comprises the step of treating the sample material with a basic solution.

The method according to the present invention allows to determine the number of the germinable cells in the sample, the concentration of the germinable cells in the sample or both. The determination of the number of cells has the advantage that it does not need a reference base such as a specific volume or mass and is therefore somewhat easier to determine. However, when the result is obtained in step d) as the number of cells and shall be further used in the determination of other parameters, see embodiment below, one has to use the same volume or the same mass of the sample in question in all determinations. Alternatively, one can already determine the concentration of the germinable cells in the sample, which has the advantage that one already has determined the cell number, relative to a reference base. This facilitates the further use of the result obtained in step e) in other embodiments and allows for an easier comparison of the thus obtained results. In the context of the present invention, preference is therefore given to the determination of cell concentration(s). The concentration of cells can be given as copies/g or copies/μL.

Generally, the pre-treatment of step a) aims to an improved differentiation between germinable cells and all other cells. It was found that the lysis of the vegetative cells in the pre-treatment of step a) leads to a significant increase in preciseness of the quantitative polymerase chain reaction of step d).

In principle, the lysis of the vegetative cells in the pre-treatment of step a) is not subject to any limitation. Rather, it can be achieved by any conceivable treatment which leads to the lysis of the vegetative cells. However, it was found that a treatment of the sample material with a basic solution either alone or in combination with an elevated temperature or an additional mechanic treatment leads to an effective lysis of the vegetative cells. The basic solution leads to a denaturing of the vegetative cells. In detail, these conditions lead to a disturbed wall structure or a damaged cell wall structure of the vegetative cells, including the cortex region and the inner membrane wall. However, due to a high resistance to bases and/or a high heat resistance some cells, specifically germinable cells, can survive these harsh conditions.

In an embodiment of the method according to the present invention the step a) further comprises subjecting the sample material to an elevated temperature and/or an additional mechanic treatment.

In principle, the pre-treatment of step a) is not subject to any limitations regarding a specific basic solution or the strength of the basic solution, provided that the basic solution either alone or in combination with additional heating and/or additional mechanic treatment leads to the lysis of the vegetative cells. Nevertheless, it is preferred that the basic solution used in step a) has a pH of at least 8, of at least 8.5, or at least 12. For example, the basic solution is an aqueous sodium hydroxide or potassium hydroxide solution having a concentration of 0.05 mol/L. For example, said basic solution has a pKb of 0 or less, in order to ensure that the pre-treatment of step a) alone already leads to the desired lysis of the vegetative cells. Preferred bases in the basic solution of step a) are sodium hydroxide, and/or potassium hydroxide. The pre-treatment of step a) is not subject to any limitation regarding the time period in which the sample material is treated with the basic solution, said treatment may take from 10 minutes to 1 hour, from 10 minutes to half an hour, or from 10 to 15 minutes.

In a preferred embodiment of the method according to the present invention the base in the basic solution of step a) has a pKb of 0 or less.

Also, the additional step of heating the sample to an elevated temperature or additional mechanic treatment is in principle not subject to any limitations regarding its specific temperature, provided that the elevated temperatures contribute to the lysis of the vegetative cells. Nevertheless, it is preferred that the elevated temperature of the additional heating of the sample is a temperature of at least 80 °C because this temperature contributes to make the lysis of the vegetative cells even more efficient.

The additional mechanic treatment can be a mixing or shaking, such as an orbital shaking, where appropriate in the presence of beads.

In another preferred embodiment of the method according to the present invention the elevated temperature of the additional heating is a temperature of at least 80 °C.

The elevated temperature of the additional heating, in particular the temperature of at least 80 °C, is applied for a time period of from 10 minutes to 1 hour, from 10 minutes to half an hour, or from 10 to 15 minutes. Likewise, the additional mechanic treatment is applied for a time period of from 10 minutes to 1 hour, from 10 minutes to half an hour, or from 10 to 15 minutes.

In order to improve the preciseness of the qPCR in step b) of the method according to the present invention, a photoreactive DNA-binding dye is added to the pre-treated sample material obtained from step a). Preferably, the addition of the photoreactive DNA-binding dye is preceded by protecting the pre-treated sample material from light. After addition of the photoreactive DNA-binding dye, the sample material is subjected to the photolysis.

In one embodiment of the method according to the present invention the step b) comprises the steps of
b1) protecting the pre-treated sample material obtained from step a) from light,
b2) adding a photoreactive DNA-binding dye to the sample material of step b1), and
b3) photolyzing the sample material obtained from step b2).

This procedure aims to inhibit the amplification of all the cells with a disturbed wall structure or a damaged cell wall structure including the cortex region and the inner membrane wall. While dead cells already have a disturbed wall structure or a damaged cell wall structure, the pre-treatment of step a) leads to a lysis of vegetative cells, specifically to a damage of the cell wall structure of vegetative cells. The photoreactive DNA-binding dye can intercalate or penetrate into the cells with a damaged cell wall structure including the cortex region and the inner membrane wall and upon photolysis the photoreactive DNA-binding dye binds to the DNA of these cells. This process renders the DNA insoluble and results in its loss during subsequent genomic DNA extraction. Consequently, the DNA of the cells with a disturbed wall structure or a damaged cell wall structure including the cortex region and the inner membrane wall is not amplified in a quantitative polymerase chain reaction. In contrast, the DNA of cells with an intact cell wall structure is amplified in a quantitative polymerase chain reaction.

The photolysis is preferably performed for a time period of from 10 to 25 minutes, or from 10 to 20 minutes, for example 10, 11, 12, 13, 14 or 15 minutes.

The irradiation wavelength in the photolysis, i.e., step b3), depends on the wavelength of the absorption maximum of the photoreactive DNA-binding dye added to the sample material. For example, the photoreactive DNA-binding dye propidium monoazide (PMA) has an absorption maximum at a wavelength of 464 nm before reacting with the DNA of the cells, and after the reaction the PMA bound to the DNA of the cells has an absorption maximum at a wavelength of 510 nm and an emission maximum at a wavelength of 610 nm.

In another embodiment of the method according to the present invention the photoreactive DNA-binding dye intercalates or penetrates into the DNA of cells with a disturbed wall structure and/or cells with a damaged cell wall structure including the cortex region and the inner membrane wall.

In principle, the step b) is not subject to any limitations regarding a specific photoreactive DNA-binding dye, provided that the dye is photoreactive and binds to DNA in a photolysis. Nevertheless, it is preferred that the photoreactive DNA-binding dye is propidium monoazide, ethidium bromide monoazide, a derivative of any of these, and/or a mixture thereof since these dyes have proven to bind very efficiently to the DNA of cells with a damaged cell wall structure including the cortex region and the inner membrane wall.

In a further embodiment of the method according to the present invention the photoreactive DNA-binding dye is propidium monoazide, ethidium bromide monoazide, a derivative of any of these, and/or a mixture thereof.

Preferably, the photoreactive DNA-binding dye is propidium monoazide (PMA). PMA is a fluorescent and photoreactive DNA-binding dye that preferentially binds to double-stranded DNA. The cells are mixed with PMA so that the dye enters the membrane of the cells. Visible light (high power halogen lamps or specific LED devices) induces a photoreaction of the chemical that will lead to a covalent bond with PMA and the dsDNA. In detail, when exposed to light, photolysis of the PMA causes the azide group of PMA to convert to a nitrene radical, which covalently binds to any carbon in close proximity, including double-stranded DNA. Theoretically, dead microorganisms lose their capability to maintain their membranes intact, which leaves the “naked” DNA in the cytosol ready to react with PMA. DNA of living organisms are not exposed to the PMA, as they have an intact cell membrane. After treatment with the chemical, only the DNA from living bacteria is usable in qPCR, allowing to obtain only the amplified DNA of living organisms.

In principle, the method according to the present invention is not subject to any limitations regarding the concentration in the which photoreactive DNA-binding dye is added to the same material in step b2). Nevertheless, it was found that the method according to the present invention even allows for a rather precise determination of germinable cells, when the photoreactive DNA-binding dye is used in a relatively low or even very low concentration, such as 50 or even only 20 micromolar (μM). It is therefore preferred to use the photoreactive DNA-binding dye in a concentration of up to 50 micromolar (μM) or in a concentration of at least 20 micromolar (μM). It is further preferred to use the photoreactive DNA-binding dye in a concentration of from 20 to 50 micromolar (μM).

According to the present invention the nucleic acids are separated from the photolyzed sample material obtained from step b) and then subjected to a quantitative polymerase chain reaction to obtain the number of germinable cells in the sample of step a). The reaction of the photoreactive DNA-binding with the DNA of the cells with a disturbed wall structure or a damaged cell wall structure including the cortex region and the inner membrane wall renders this DNA insoluble. This facilitates the separation of the DNA of the germinable cells for the following quantitative polymerase chain reaction.

In principle, the separation of step c) is not subject to any limitations, and can, in principle, be done in any conceivable manner, provided it allows for a separation of the DNA of the germinable cells as complete as possible.

In order to conduct nucleic acid separation, it is necessary to release nucleic acids from the germinable cells as complete as possible, which is preferably achieved by subjecting the photolyzed sample material obtained from step b) to a lysis, releasing proteins, DNA, phospholipids, etc. from the cells. The remaining tissue is discarded. Said lysis is a chemical and/or a mechanical lysis. As already mentioned above, the photolysis of step b) above leads to the reaction of the photoreactive DNA-binding dye to the DNA of the cells with a disturbed wall structure or a damaged cell wall structure including the cortex region and the inner membrane wall, which results in the insolubility of the DNA of the non-viable cells. Therefore, one can expect that the DNA of the non-viable cells is discarded together with the tissue. The further separation of the nucleic acids of the germinable cells involves the binding of the nucleic acids released in and obtained from the said lysis to beads to obtain loaded beads. The beads can be made of silica or zirconia. In order to remove any potential contaminations which may still be attached to the beads, it is preferred that the loaded beads are washed. Next, the nucleic acids are released again from the washed beads, preferably by treatment with an elution buffer, which gives a solution comprising the released nucleic acids.

In yet another embodiment of the method according to the present invention the step c) comprises the steps of
c1) subjecting the photolyzed sample material obtained from step b) to a chemical and/or mechanical lysis to release nucleic acids from the sample material,
c2) binding the released nucleic acids obtained from step c1) to beads to obtain loaded beads,
c3) washing the loaded beads obtained from step c2), and
c4) releasing the nucleic acids from the washed beads obtained from step c3) by treatment with an elution buffer or water to obtain a solution comprising the released nucleic acids.

In the context of the present invention, it is preferred that the beads are magnetic which facilitates the separation of the loaded beads from the solution, suspension or dispersion obtained from the chemical and/or mechanical lysis of the photolyzed sample material and their transfer to a different container for washing and release of the nucleic acids.

In principle, the chemical lysis of the photolyzed cells in step c1) is not subject to any limitations. Rather, it can be achieved by any conceivable chemical treatment which leads to the lysis of the cells. However, it was found that a chemical lysis of the photolyzed sample material under chaotropic conditions either alone or in combination with the mechanical lyses leads to an effective lysis of the cells. It is therefore preferred that the chemical lysis of step c1) is performed under chaotropic conditions.

Chaotropic conditions lead to a disruption of the structure of, and denaturation of, macromolecules such as proteins and nucleic acids (e.g., DNA and RNA). Chaotropic solutes increase the entropy of the system by interfering with intermolecular interactions mediated by non-covalent forces such as hydrogen bonds, van der Waals forces, and hydrophobic effects. Macromolecular structure and function are dependent on the net effect of these forces. Therefore, it follows that an increase in chaotropic solutes in a biological system will denature macromolecules, reduce enzymatic activity and induce stress on a cell (i.e., a cell will have to synthesize stress protectants). Tertiary protein folding is dependent on hydrophobic forces from amino acids throughout the sequence of the protein.

Chaotropic solutes decrease the net hydrophobic effect of hydrophobic regions because of a disordering of water molecules adjacent to the protein. This solubilizes the hydrophobic region in the solution, thereby denaturing the protein. This is also directly applicable to the hydrophobic region in lipid bilayers; if a critical concentration of a chaotropic solute is reached (in the hydrophobic region of the bilayer) then membrane integrity will be compromised, and the cell will lyse.

Common chaotropic agents include n-butanol, ethanol, guanidinium chloride, lithium perchlorate, lithium acetate, magnesium chloride, phenol, 2-propanol, sodium dodecyl sulfate, thiourea, and urea.

Chaotropic salts that dissociate in solution exert chaotropic effects via different mechanisms. Whereas chaotropic compounds such as ethanol interfere with non-covalent intramolecular forces as outlined above, salts can have chaotropic properties by shielding charges and preventing the stabilization of salt bridges. Hydrogen bonding is stronger in non-polar media, so salts, which increase the chemical polarity of the solvent, can also destabilize hydrogen bonding. Mechanistically this is because there are insufficient water molecules to effectively solvate the ions. This can result in ion-dipole interactions between the salts and hydrogen bonding species which are more favorable than normal hydrogen bonds. It is therefore preferred that the chemical lysis of step c1) is performed under chaotropic conditions, in particular, under chaotropic high salt conditions.

In a preferred embodiment of the method according to the present invention wherein the chemical lysis of step c1) is performed under chaotropic conditions.

In principle, the chemical lysis of the photolyzed cells in step c1) is not subject to any limitations. Rather, it can be achieved by any conceivable chemical treatment which leads to the lysis of the cells. Typically, in mechanical lysis, cell membrane is physically broken down by using shear force. This method is the most popular and is available commercially because of a combination of high throughput and higher lysing efficiency. Examples for mechanical lysis techniques include the use of the high-pressure homogenizers, and bead mills.

High-Pressure Homogenizer (HPH) is one of the most widely used equipment for large scale microbial disruption. In this method, cells in media are forced through an orifice valve using high pressure. Disruption of the membrane occurs due to high shear force at the orifice when the cell is subjected to compression while entering the orifice and expansion upon discharge.

Bead mill, also known as bead beating method, is a widely used laboratory scale mechanical cell lysis method. The cells are disrupted by agitating tiny beads made of glass, steel or ceramic which are mixed along with the cell suspension at high speeds. The beads collide with the cells breaking open the cell membrane and releasing the intracellular components by shear force. This process is influenced by many parameters such as bead diameter and density, cell concentration and speed of agitator. Smaller beads with a diameter in the range of from 0.25-0.5 mm are more effective and thus recommended for lysis.

According to the present invention the released nucleic acids obtained from step c1) are bound to beads to obtain loaded beads. It is therefore preferred that the mechanical lysis of step c1) is already performed using beads, i.e., using the same beads as in step c2).

The advantage of using beads in the mechanical lysis of step c1) and in the binding of step c2) is that both steps can be performed at one or at least with a high degree of overlap in time. For example, when the silica-based beads are used in step c1), one can perform a nucleic acid separation by silica adsorption. This method of DNA separation that is based on DNA molecules binding to silica surfaces in the presence of certain salts and under certain pH conditions. In order to conduct DNA separation by silica adsorption, a sample (this may be anything from purified cells to a tissue specimen) is lysed, releasing proteins, DNA, phospholipids, etc. from the cells. The remaining tissue is discarded. The supernatant containing the DNA is then exposed to silica in a solution with high ionic strength. The highest DNA adsorption efficiencies occur in the presence of buffer solution with a pH at or below the pKa of the surface silanol groups.

The mechanism behind DNA adsorption onto silica is not fully understood; one possible explanation involves reduction of the silica surface's negative charge due to the high ionic strength of the buffer. This decrease in surface charge leads to a decrease in the electrostatic repulsion between the negatively charged DNA and the negatively charged silica. Meanwhile, the buffer also reduces the activity of water by formatting hydrated ions. This leads to the silica surface and DNA becoming dehydrated. These conditions lead to an energetically favorable situation for DNA to adsorb to the silica surface.

A further explanation of how DNA binds to silica is based on the action of guanidinium chloride (GuHCI), which acts as a chaotrope. A chaotrope denatures biomolecules by disrupting the shell of hydration around them. This allows positively charged ions to form a salt bridge between the negatively charged silica and the negatively charged DNA backbone in high salt concentration. The DNA can then be washed with high salt and ethanol, and ultimately eluted with low salt.

After the DNA is bound to the silica it is then washed to remove contaminants and finally eluted using an elution buffer or distilled water. The elution buffer can be any suitable buffer which allows for solubilizing DNA, while protecting it from degradation. A suitable elution buffer is for example, TE buffer, which comprises Tris, a common pH buffer, and EDTA, a molecule that chelates cations like Mg²⁺. For example, EDTA further inactivates DNase, by binding to metal cations required by this enzyme, and thus protects the DNA from degradation.

According to the present invention the separated nucleic acids obtained from step d) are subjected to a quantitative polymerase chain reaction (qPCR) to obtain the number of germinable cells in the sample of step a).

The qPCR of the method according to the present invention uses nucleotides, i.e., primers forward primers reverse, probes, and quantification vectors, which were synthesized for the quantification of the targets of interest, and which therefore are specific for the bacterial strains in question.

In principle, the quantitative polymerase chain reaction of step d) is not subject to any limitations, provided that it allows to obtain the number and/or concentration of germinable cells. The term quantitative polymerase chain reaction, or qPCR in short, is used as known to the person skilled in the art, and denotes a method widely used to rapidly make millions to billions of copies (complete or partial) of a specific DNA sample, allowing scientists to take a very small sample of DNA and amplify it (or a part of it) to a large enough amount to study in detail. In principle, a qPCR is a DNA amplification reaction of the target DNA, in this context also referred to as template DNA. A typical amplification reaction includes a target DNA, a thermostable DNA polymerase, two oligonucleotide primers, deoxynucleotide triphosphates (NTPs), reaction buffer, and magnesium. Once the reaction mixture is combined, it is placed in a so-called cycler, an instrument that subjects the reaction mixture to a series of different temperatures for set amounts of time. This series of temperature and time adjustments is referred to as one cycle of amplification. Each qPCR cycle theoretically doubles the amount of target (amplicon) in the reaction. Ten cycles theoretically multiply the amplicon by a factor of about one thousand and 20 cycles, by a factor of more than a million in a matter of hours.

Each cycle of qPCR includes steps for template denaturation, primer annealing and primer extension. The initial step denatures the target DNA by heating it to an elevated temperature, e.g., to a temperature of ca. 95 °C for 15 seconds to two minutes. In the denaturation step, the two intertwined strands of a double-stranded DNA (dsDNA) separate from one another, producing the single-stranded DNA necessary for replication by the thermostable DNA polymerase. In the next step of a cycle, the annealing step, the temperature is reduced to a temperature in the range of from 40 to 60 °C. At a temperature in this range, the oligonucleotide primers can form stable associations with the denatured target DNA, in other word they anneal with the single-stranded DNAs and serve as primers for the DNA polymerase. This step lasts for approximately 15 to 60 seconds. The third step of the qPCR cycle, the extension step, begins as the reaction temperature is raised again to a temperature in the range of from 70 to 74 °C. This is the optimum temperature range for most thermostable DNA polymerases. The extension step lasts for approximately 1 to 2 minutes. The next cycle begins with a return to a temperature of ca. 95 °C for denaturation.

Each step of the cycle should be optimized for each template and primer pair combination. If the temperature during the annealing and extension steps are similar, these two steps can be combined into a single step in which both primer annealing and extension take place. In the context of the present invention, the amplified product is analyzed for quantity after 20 to 40 cycles.

The specific temperatures and durations for each step of the cycle in the qPCR depends on the target DNA in question. Therefore, the qPCR in step d) of the method according to the present invention is not subject to any limitations. Nevertheless, it is preferred that the denaturation is performed at a temperature of up to 95 °C for a duration of 3 minutes+/−1 minute, the annealing step is performed at a temperature of ca. 55 °C for a duration of 30 seconds+/−10 seconds, and the amplification step is performed at a temperature of up to 95 °C for a duration of ca. 15 seconds+/−10 seconds. Preferably, the whole cycling time is up to ca. 1 hour and 18 minutes+/−10 minutes.

Quantitative PCR (qPCR) allows the quantification and detection of a specific DNA sequence in real time since it measures concentration while the synthesis process is taking place.

In a quantitative polymerase chain reaction, relative fluorescence units (RFU) measurements are used for DNA profiling. The photolysis of step b) leads to a DNA probe consisting of nucleic acids that are labeled with the fluorescent dye which allows for the detection after hybridization with the complementary DNA target. A thermal cycler equipped with fluorescence detection units is used to measure the fluorescence real time during the amplification of the target genes. The amount of measured fluorescence (expressed as RFU) reflects the amount of amplified product in each cycle. For the quantification of the target, the quantification cycle (Cq), at which the fluorescence reaches a detectable signal is determined. The Cq is proportional to the initial concentration of the target gene at the start of the amplification. A computer program measures the results, determining the quantity or size of the fragments, at each data point, from the level of fluorescence intensity. The quantitative polymerase chain reaction in step d) of the method according to the present invention therefore gives the concentration of germinable cells in the sample of step a).

Based on this result, it is also possible to determine the number and/or concentration of the other cells, i.e., the non-viable and the vegetative cells, in the sample of step a). For this purpose, a part of the sample material of step a) is subjected to a determination of the number and/or concentration of all cells in the sample material, followed by taking the difference between the thus obtained number and/or concentration of all cells and the number and/or concentration of germinable cells in the sample material of step a). The thus obtained parameter is therefore a sum parameter.

In a further embodiment the method according to the present invention further comprises the steps of
e) determining the number and/or concentration of non-viable and vegetative cells in the sample of step a), comprising the steps of
   e1) subjecting a part of the sample material of step a) to a determination of the number and/or concentration of all cells in said sample material, and
   e2) taking the difference between the number and/or concentration of all cells obtained in step e1) and the number and/or concentration of germinable cells obtained in step d) to give the number and/or concentration of the non-viable cells and the vegetative cells in the sample material of step a).

In principle, step e1) is not subject to any limitation regarding the procedure for determining the number and/or concentration of all cells, provided that the procedure in question allows for a reliable and accurate determination of the number and/or concentration of all cells in the sample of biological origin. For example, microbial counting or qPCR is suitable for the determination in step e1). In any case, the determination of the number and/or concentration of all cells is done in the absence of a DNA binding dye in the sample material. In the context of the present invention, it is preferred to perform step e1) using qPCR since it is already used in step d) and it allows the determination of the value(s) of interest with the desired precision.

When the number of the germinable cells was determined in step d), the step e1) uses the same volume and/or mass of the sample material as used in step d). When the concentration of the germinable cells was determined in step d), the step e1) uses the same type of concentration, meaning either copies/g or copies/μL, as used in step d).

In case of interest, the method according to the present invention also allows for the determination of the percentage of the number of germinable cells obtained in step d), relative to the number of all cells obtained in step e1) and/or the percentage of the number of non-viable and vegetative cells obtained in step e2), relative the number of all cells obtained in step e1).

In a preferred embodiment the method according to the present invention further comprises the step of
f) determining the number and/or concentration of the vegetative cells in the sample of step a), comprising the steps of
   f1) subjecting a part of the sample material of step a) to a determination of the number and/or concentration of the viable cells in said sample material, and
   f2) taking the difference between the number and/or concentration of the viable cells obtained in step f1) and the number and/or concentration of the germinable cells obtained in step d) to give the number and/or concentration of the vegetative cells in the sample material of step a).

In principle, step f1) is not subject to any limitation regarding the procedure for determining the number and/or concentration of viable cells, provided that the procedure in question allows for a reliable and accurate determination of the number and/or concentration of viable cells in the sample of biological origin. In the context of the present invention, it is preferred to perform step f1) using qPCR, wherein the sample material is treated with a DNA binding dye prior to the qPCR.

When the number of the germinable cells was determined in step d), the step f1) uses the same volume and/or mass of the sample material as used in step d). When the concentration of the germinable cells was determined in step d), the step f1) uses the same type of concentration, meaning either copies/g or copies/μL, as used in step d).

Further, the method according to the present invention also allows for the determination of the change in the number and/or concentration of germinable cells in the sample in question, relative to the number or concentration of all cells, based on the results obtained in steps d) and e1). This measure for a change describes the change between the “before” and “after” values of a quantity. Here, the percentage of the relative change is used. The percentage change is equal to the quotient of the absolute change and the baseline value multiplied by 100%. The percentage change is therefore a relative change in percentage notation without a physical unit. The basic value y1 is also the 100% value. The percentage change describes in percent how much a given base value has changed, i.e., increased or decreased. The number or concentration of cells in the sample of interest has not changed over time, However, the number of the individual types of cells has changed. Specifically, the number of germinable cells has decreased, a negative value for the percentage relative change therefore reflects the degree of decrease in the number and/or concentration of germinable cells.

In another preferred embodiment the method according to the present invention further comprises the step of
g) determining the relative change in the number and/or concentration of germinable cells, relative to the number of all cells, by means of the formula

$$rel . ( \% ) = \frac{{n}_{g} - {n}_{all}}{{n}_{all}} \times 100 and / or rel . ( \% ) = \frac{{c}_{g} - {c}_{all}}{{c}_{all}} \times 100$$

wherein
   rel. (%) is the relative change in the number and/or concentration of germinable cells, in percent,
   n_{g} is the number of germinable cells, obtained in step d),
   c_{g} is the concentration of germinable cells, obtained in step d),
   nₐₗₗ is the number of all cells, obtained in step e1), and
   cₐₗₗ is the concentration of all cells, obtained in step e1).

In principle, the method according to the present invention is not subject to any limitations regarding a specific sample of biological origin or regarding a sample of a specific biological origin.

Nevertheless, the method according to the present invention is particularly suitable for samples of biomass, feed, fecal, or digesta.

In a further embodiment of the method according to the present invention the sample of step a) is a biomass, feed, fecal, or digesta sample.

In a preferred embodiment of the method according to the present invention the sample of step a) is a probiotic. Preferably, the sample is a probiotic comprising a Bacillus spp., for example a B. amyloliquefaciens and/or B. subtilis, such as Ecobiol®, Gutcare®, and/or GutPlus® of Evonik.

The qPCR of the method according to the invention uses nucleotides, i.e., primers forward, primers reverse, probes, and quantification vectors, which were sequenced for the quantification of the targets of interest, and which therefore are specific for the bacterial strains in question. It is therefore preferred that nucleotides used in this qPCR, i.e., primers forward, primers reverse, probes, and quantification vectors were sequenced for the quantification of a Bacillus spp., for example a B. amyloliquefaciens and/or B. subtilis.

### FIGURES

FIG. 1a shows the correlation of the microbiological spore count between Lab1 (external laboratory) and Lab2 (QA laboratory Evonik)

FIG. 1b is a box plot representation of the number of colony-forming units (CFU) of Bacillus spp. determined in the interlaboratory ring trial test between the participating laboratories (N=13), wherein the indication of the participating laboratories was randomized in order to anonymize the individual laboratories.

FIG. 2a shows the linear regression between the values measured with the method according to the present invention and with microbial spore count, where the measurements were correlated with Lab1.

FIG. 2b shows the linear regression between the values measured with the method according to the present invention and with microbial spore count, where the measurements were correlated with Lab2.

FIG. 3a shows the evaluation of the different sample pre-treatments without the addition of the DNA binding dye propidium monoazide (PMA) for the GutCare® feed sample DE22-006 with the strain Bacillus subtilis DSM 32315.

FIG. 3b shows the linearity of the PMA concentrations over a range of from 0 μM to 120 μM.

FIG. 4 illustrates the number of germinable cells for the strain Bacillus subtilis DSM 32315 determined with the method according to the present invention with different pre-treatments in comparison to the microbial spore count for the GutCare® feed sample DE22-006.

FIG. 5 illustrates the number of germinable cells for the strain Bacillus subtilis DSM 32540 determined with the method according to the present invention with different pre-treatments in comparison to the microbial spore count for the GutPlus® feed sample DE22-007.

FIG. 6 illustrates the number of germinable cells for the strain Bacillus amyloliquefaciens CECT 5940 determined with the method according to the present invention with different pre-treatments in comparison to the microbial spore count for the Ecobiol® feed sample DE22-008.

FIG. 7 illustrates the number of germinable cells for the combination of the strain Bacillus subtilis DSM 32315 (GutCare®) with the strain Bacillus subtilis DSM 32540 (GutPlus®) determined with the method according to the present invention with different pre-treatments in comparison to the microbial spore count for the DE22-001 feed sample.

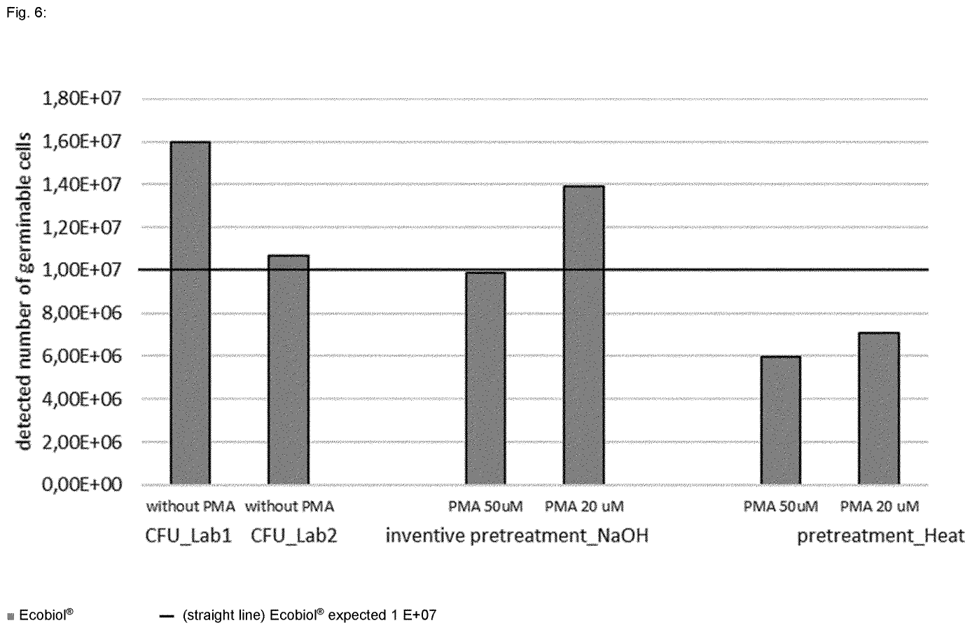

FIG. 6 illustrates the number of germinable cells for the combination of the strain Bacillus subtilis DSM 32315 (GutCare®) with the strain Bacillus amyloliquefaciens CECT 5940 (Ecobiol®) determined with the method according to the present invention with different pre-treatments in comparison to the microbial spore count for the DE22-004 feed sample.

FIG. 9 illustrates the number of germinable cells for the combination of the strain Bacillus subtilis DSM 32540 (GutPlus®) with the strain Bacillus amyloliquefaciens CECT 5940 (Ecobiol®) determined with the method according to the present invention with different pre-treatments in comparison to the microbial spore count for the DE22-003 feed sample.

FIG. 10 shows the results for the Ecobiol® feed sample DE22-008 with the single strain Bacillus amyloliquifaciens CECT 5940 determined with the method according to the present invention without PMA and with different PMA concentrations when pre-treated with pure water.

FIG. 11 shows the results for the Ecobiol® feed sample DE22-008 with the single strain Bacillus amyloliquifaciens CECT 5940 determined with the method according to the present invention without PMA and with different PMA concentrations when pre-treated with NaOH.

FIG. 12 shows the results for the Ecobiol® feed sample DE22-008 with the single strain Bacillus amyloliquifaciens CECT 5940 determined with the method according to the present invention without PMA and with different PMA concentrations when pre-treated with heat.

FIG. 13 shows the linear regression between the values determined with the method according to the present invention and with microbial spore count, in which the measurements were correlated with CFU Lab2, when 50 μM of PMA were applied.

FIG. 14 shows the linear regression between the values determined with the method according to the present invention and with microbial spore count, in which the measurements were correlated with CFU Lab2, when 20 μM of PMA were applied.

### EXPERIMENTS

### 1. Pre-Treatment and Photolysis

### 1.1 Consumables and Materials Needed

100-1000 μl pipette
sterile filter tips for 100-1000 μl pipette
10-100 μl pipette
sterile filter tips for 10-100 μl pipette
0.5-10 μl pipette
sterile filter tips for 0.5-10 μl pipette
1.5 ml tubes
racks for 1.5 ml tubes
disposable gloves
waste bags
propidium monoazide (PMA)
zirconia glass pellets
aluminum foil
0.9% NaCl
ultrapure water
ScreenFloX® qPCR stabilizer buffer

### 1.2 Devices Needed

BagMixer (Interscience)
PMA Lite™ LED photolysis device (E90002, Biotium)
centrifuge for 1.5 ml centrifuge tubes with a speed of up to 5000 rcf (relative centrifugal force)
MillMix20
Eppendorf thermomixer (heat bath)

### 1.3 Pre-Treatment of the Sample Material

1.3.1 Pre-Treatment of the Sample Material with Sodium Hydroxide
A sodium hydroxide stock solution (c(NaOH)=0.05 mol/L) was prepared by dissolving 2 g of sodium hydroxide in 1 L of ultrapure water.
5 g of the sample material, e.g., feeding stuff comprising bacilli spores, were weighed in a stomacher bag, and sodium hydroxide stock solution was added to a final weight of 100 g.
The filled stomacher bag was clamped inside a stomacher device (BagMixer, Interscience) and shaken for 5 minutes at a speed level of 3. The shaking led to a homogenization step within the stomacher device for 5 minutes. A pH of at least 8.5 in the initial solution ensured that the absorption of the bacilli spores to the feeding stuff was kept to a minimum. Dilution with the sodium hydroxide stock solution destroyed the vegetative cells in the bag.
After the homogenization step the thus obtained suspension was transferred into a Duran® bottle.
10 mL of the thus obtained sample solution were pipetted into a 50 mL centrifuge tube, and the tube was tightly closed.
   1.3.2 Pre-Treatment of the Sample Material with Heat Treatment
5 of the sample material, e.g., feeding stuff comprising bacilli spores, were weighed in a stomacher bad and filled up with 100 mL of ultrapure water.
The filled stomacher bag was clamped inside a stomacher device (BagMixer, Interscience) and shaken for 5 minutes at a speed level of 3. This led to a homogenization step within the stomacher device for 5 minutes.
After the homogenization step the thus obtained suspension was transferred into a Duran® bottle.
10 mL of the thus obtained sample solution were pipetted into a 50 mL Falcon tube, and the tube was tightly closed.
The sample tube was incubated for 10 minutes in a pre-tempered water bath having a temperature of 80 °C.
After the incubation the tube cooled for minutes in an ice bath.

### 1.4 PMA Coloring and Preparing for the Extraction Process

The suspension obtained in 1.3.1 and/or 1.3.2 were aliquoted. For this purpose, 4 aliquots with each 1 mL were taken from each suspension and fractionated into tubes of 4 mL.
The thus obtained fractionated samples were protected from light and this protection was maintained for whole PMA coloring procedure.
A stock solution of PMA having a concentration of 20 mM was provided.
2.5 μl or 1 μl of said stock solution were added to the light protected fractionated samples to give a concentration of 50 μM (2.5 μl) or 20 μM (1 μl).
The lighted protected fractionated samples with PMA were incubated at 10 °C under shaking at 1200 rpm (20 Hz) for 10 minutes.
Next, the samples were incubated for 15 minutes in the PMA Lite (photolysis) at an LED output wavelength of 465 to 475 nm.
During the illumination phases the tubes were taken out of the PMA lite every 4 minutes and shortly shaken or mixed to guarantee a good illumination at each point.
All sample tubes were centrifuged at 10,000 rpm for 10 minutes.
The supernatant was removed with a pipette from each tube and discarded.
1 mL Lysis buffer from the ScreenFloX® sample collection tube and 5 zirconia beads were added to each tube.
Next, the content of the tubes was mixed in a Millmix at 20 Hz for 10 minutes.
After the mixing, the tubes were tempered in a heat bad at 85 °C for 20 minutes.
After tempering, the content of the tubes was mixed again in a Millmix at 20 Hz for 10 minutes.
Then, the content of the tubes was subjected to the extraction and qPCR procedure.

### 2. Extraction

### 2.1 Consumables and Materials Needed

100-1000 μl pipette
Sterile filter tips for 100-1000 μl pipette
Multipette® E3
Eppendorf Combitips advanced®, Biopur®, 0.2 ml, lightblue, biopur (single blistered)
Eppendorf Combitips advanced®, Biopur®, 10 ml, lightblue, biopur (single blistered)
KingFisher Deep Well plates
KingFisher Microtiterplates
KingFisher Tip Combs
Foil for sealing DNA eluate plates (should be easy to remove)
Rack for 1.5 ml and 2 ml tubes
Disinfectant for bench and KingFisher cleaning
Canister for liquid waste disposal
Funnel for liquid disposal from KingFisher plates
Waste bags
Disposable gloves
ScreenFloX® Nucleic acid extraction kit
Internal DNA extraction control kit VIC-channel (PrimerDesign)

### 2.2 Devices Needed

KingFisher Flex (ThermoFisher)
Vortex mixer
Centrifuge 5425 with rotor FA-24×2 (Eppendorf)

### 2.3 Method

The “ScreenFloX®_AnsExtraction” protocol was selected on the KingFisher Flex and “Start” was pressed. The instructions on the KingFisher display were followed.
The “Comb plate” was prepared by adding a 96-tip comb into an empty deep well 96-plate, the plate was placed into the instrument on the loading position, and start was pressed to change proceed with the loading.
The “Eluate plate” (KingFisher™ 96 μlate 200 μl) was prepared by adding 80 μl elution buffer into each well used, the plate was placed into the instrument on the loading position, and start was pressed to change proceed with the loading.
The “Wash 3 Plate” (KingFisher™ 96 deep well plate) was prepared by adding 500 μl wash buffer 3 into each well used, the plate was placed into the instrument on the loading position, and start was pressed to change proceed with the loading.
The “Wash 2 Plate” (KingFisher™ 96 deep well plate) was prepared by adding 500 μl wash buffer 2 into each well used, the plate was placed into the instrument on the loading position, and start was pressed to change proceed with the loading.
The “Wash 1 Plate” (KingFisher™ 96 deep well plate) was prepared by adding 500 μl wash buffer 1 into each well used, the plate was placed into the instrument on the loading position, and start was pressed to change proceed with the loading.
The “Sample Plate” was prepared by adding 850 μl of the sample (from the PMA staining), 25 μl magnetic beads, 20 μl enhancer and 4 μl resuspended internal control (lyophilized internal control DNA template from PrimerDesign) into each well, the plate was placed into the instrument on the loading position, and “Start” was pressed to start the nucleic acid extraction.

When reloading the KingFisher™ Flex, the instructions on the display were followed. When not proceeding directly with the qPCR, the eluate plate was sealed and stored at 4 °C for a short time and at −20 °C for a long time. A repeated thawing and freezing of the eluate plate was avoided.

### 3. qPCR Detection

### 3.1 Consumables and Material Needed

100-1000 μl pipette
sterile filter tips for 100-1000 μl pipette
10-100 μl pipette
sterile filter tips for 10-100 μl pipette
0.5-10 μl pipette
sterile filter tips for 0.5-10 μl pipette
Multipette® E3
Eppendorf Combitips advanced®, Biopur®, 0.2 ml, lightblue, biopur (single blistered)
8-channel pipette 1-10 μl
96-well qPCR plate
qPCR plate foil
Roller for sealing adhesive film
DNA removal reagent
Arm sleeves for working under the qPCR hood
Rack for 1.5 and 2 ml tubes
Eppendorf 2 ml tubes, qPCR-clean
Eppendorf 1.5 ml tubes, qPCR-clean
Tweezer for taking sterile tubes
Waste bags
Disposable gloves
Luna Universal Probe qPCR Master Mix (M3004X)
PCR-graded nuclease-free water
Internal DNA extraction control kits with VIC fluorophore (PrimerDesign)

### 3.2 Devices Needed

CFX96 Touch qPCR Cycler (BioRad)
UV cabinet/qPCR hood
Vortex mixer
Centrifuge for microtubes, 2×
Centrifuge for plates

### 3.3 Nucleotides Needed

The qPCR method used nucleotides, i.e., primers forward, primers reverse, probes, and quantification vectors, which were sequenced and synthesized for the quantification of the targets of interest, and which therefore are specific for the bacterial strains in question. Here, as shown by the example of Bacillus amyloliquefaciens (Ecobiol®). All other products of Bacillus spp. were subjected to the same measurement condition and criterion described in chapter.

Long term storage of the primer and probe stocks was done at −20 °C. Primer and probe working stocks with the following concentrations
primers: 20 μmol, dilute stock 1:5
probe: 10 μmol, dilute stock 1:10
   were stored at 4 °C for up to 2 months. Extensive freeze-thawing cycles were avoided.

For the Ecobiol® QS, the lyophilized vector was solved in the elution buffer from the ScreenFloX® Nucleic acid extraction kit and the copy number were calculated according to the following equations:

$$Total number of copies = \frac{Synthetized mass [ μg ] * 6.02 \times {10}^{23} [ \frac{molecules}{mole} ]}{Molecular weight [ \frac{μg}{mole} ]} \qquad ( 1 )$$

$$Copies per μl = \frac{Total number of copies}{Solving volume [ μl ]} \qquad ( 2 )$$

Based on the calculated copies/μl, the following quantification standards were prepared by serial dilutions in elution buffer for the respective target of interest (i.e., Ecobiol® ID-gene for the quantification of the strain Bacillus amyloliquefaciens CECT 5940):

TABLE 1
Quantification standards (QS) required for the
determination method

| Standard | Concentration |
|---|---|
| QS 4 | 1.00E+04 |
| QS 3 | 1.00E+03 |
| QS 2 | 1.00E+02 |
| QS 1 | 1.00E+01 |

For long term storage, the quantification standards were stored at −20 °C. Short term storage up to 5 weeks was done at 4 °C. Extensive freeze-thawing cycles were avoided for the stock and the diluted vectors. This component contained high copy number template and was a very significant contamination risk.

### 3.4 Method

### 3.4.1 Mastermix Setup

All reagents and samples were thawed completely, mixed (by pipetting or gentle vortexing) and centrifuged briefly before use. The workflow contained a heterologous Internal Control (IC), which was used as control of the sample preparation procedure (nucleic acid extraction) and as a qPCR inhibition control.

The Mastermix comprised the Luna Universal Probe qPCR Master Mix (M3004X), the primerforward, the primer reverse, the probe, the primer design IC mix (heterologous Internal Control), the Mastermix volume, and the pre-treated sample. The heterologous Internal Control (IC) was used as a control of the sample preparation procedure (nucleic acid extraction) and as a qPCR inhibition control.

The composition of the mastermix was calculated as indicated in the following table for the amounts of reactions needed. It was considered that each quantification standard as well as a non-template control (NTC) were to be loaded twice and an extra of approximately 10% was also considered to compensate pipetting loss.

The mastermix was set up following the steps A and B listed below, carefully taking the areas for preparation under consideration.

### 3.4.2 Programming the Cycler

The qPCR for the target of interest was performed under the following settings:

TABLE 2
CFX96 Setting for the qPCR
Setting

| Reaction Volume | 20 μl |
|---|---|
| Lid Settings | default |
| Plate Type | white |

The cycling program was set to the following:

TABLE 3
Cycling program for the qPCR

| | | Plate | Temperature | Time |
|---|---|---|---|---|
| Step | Stage | read | [°C] | [min:sec] |
| Denaturation | Hold | — | 95 | 03:00 |
| Amplification | Cycling | — | 95 | 00:15 |
| | | Yes | 95 | 00:30 |

The cycling run time was conducted for 1 h 18 min.

The targets were detected via the following fluorescent dyes:

TABLE 4
Detectors and channels for the targets of the qPCR

| Target | Detector name | Channel |
|---|---|---|
| Ecobiol ® specific DNA | Ecobiol ® | FAM |
| Internal extraction control | IC | VIC |

### 4. Interpretation of Results

Prior to calculation of the copies of Ecobiol® ID-gene in the original sample, the following points were checked:
1. The baseline thresholds were set to a specific RFU value that was set for the specific CFX96 cycler,
2. the fluorescence drift correction was activated,
3. the non-template control was checked for the presence of the internal control (IC) and the absence of other targets (if this fails, the run is invalid),
4. all negative samples were checked for the presence and Cq of the IC (if this fails, the sample is invalid),
5. the standard curve was checked for the slope (R\hat{ }2>0.95) and the qPCR efficiency (95-105%), if there was a deviation the run had to be reviewed, and
6. the end point fluorescence was checked, if it was in a certain range (range depending on cycler and fluorophore)

TABLE 5
Interpretation of results

| Detection channel | | |
|---|---|---|
| FAM | VIC | Result interpretation |
| + | +* | Ecobiol ® specific DNA detected |
| − | + | The sample does not contain detectable |
| | | amounts of Ecobiol ® specific DNA |
| − | − | qPCR inhibition or reagent failure.** |

*Detection of the Internal Control in the VIC ™ detection channel is not required for positive results either in the FAM ™ detection channel or in the Cy ®5 detection channel. A high Ecobiol ® load in the sample can lead to reduced or absent Internal Control signal.
**Repeat testing of the eluate/from the original sample or collect a new sample and repeat testing.

For Ecobiol® positive samples, the standard curve of the qPCR assay enabled the quantification of the target genes in forms of starting quantity (SQ) per μl eluate. The standard curve was automatically created by the CFX Manager software when the appropriate wells were assigned as standards or were calculated by the following equation:

Cq=m×log(N₀)+b (1)

in which
Cq=quantification cycle
m=slope
N₀=starting concentration (SQ)
b=intercept.

For determining the Ecobiol® concentration in the original sample the following formula was applied:

$$GutCare [ \frac{copies}{g} ] = \frac{SQ [ \frac{copies}{μl} ] \times 80 μl elution volume}{0.85 ml sample volume )} \times \frac{20 ml buffer + 1 g sample input}{1 g sample input}$$

### 5. Technical Specifications

5.1 Specification of the qPCR Method

### 5.1.1 Limit of Detection (LOD)

The analytical sensitivity of the assay was determined as the 95% Limit of Detection (LOD), which represents the lowest amount of DNA molecules that is detected with a probability of 95%. The LOD of the optimized qPCR assay was determined by serial testing of highly diluted quantification standard. A dilution series was tested in 20 replicates.

The 95% limit of detection was calculated and resulted for:
Ecobiol® (Bacillus amyloliquefaciens CECT 5940) with 1.57±0.20 copies/μl and a confidence interval from 1.28 to 2.15 copies/μl
GutCare® (Bacillus subtilis DSM 32315) with 1,16±0,16 copies/μl and a confidence interval from 0.92 to 1.65 copies/μl.
GutPlus® (Bacillus subtilis DSM 32540) with 1.72±0.23 copies/μl and a confidence interval from 1.38 to 2.41 copies/μl.

### 6. Results

Various products comprising different bacterial strains and a feed product were applied for microbial spore count and the method according to the present invention. The data of said products is summarized in Table 6.

TABLE 6
Data of used probiotic products, the strains contained therein,
and data of the used feed product

| Tested product | Manufacturer | Strain |
|---|---|---|
| GutCare ® | Evonik Operations GmbH | B, subtilis (DSM 32315) |
| GutPlus ® | Evonik Operations GmbH | B. subtilis (DSM 32540) |
| Ecobiol ® 500 | Evonik Operations GmbH | B. amyloliquefaciens |
| | | (CECT 5940) |
| Broiler Feed | Raiffeisen GmbH | — |

Samples with different combinations of the products of Table 6 were prepared. Further, the expected concentrations for the strains of the individual products and the expected microbial spore count in colony forming units (CFU) for the resulting combinations were estimated.

TABLE 7
Composition of the samples and the microbial spore counts.

| | Microbial | Microbial | Microbial | Microbial spore | |
|---|---|---|---|---|---|
| | spore | spore | spore count | count | |
| | count | count | Ecobiol ® | (expected sum | |
| | GutCare ® | GutPlus ® | 500 | of all products) | Product |
| Lab-ID | [CFU/g] | [CFU/g] | [CFU/g] | [CFU/g] | combination |
| DE22-001 | 1.0E+07 | 1.0E+07 | — | 2.0E+07 | GutCare ® + GutPlus ® |
| DE22-002 | 1.0E+07 | — | 1.0E+07 | 2.0E+07 | GutCare ® + Ecobiol ® |
| DE22-003 | — | 1.0E+07 | 1.0E+07 | 2.0E+07 | GutPlus ® + Ecobiol ® |
| DE22-004 | 1.0E+07 | | 1.0E+06 | 1.1E+07 | GutCare ® + Ecobiol ® |
| DE22-005 | — | 1.0E+07 | 1.0E+06 | 1.1E+07 | GutPlus ® + Ecobiol ® |
| DE22-006 | 1.0E+07 | | — | 1.0E+07 | GutCare ® |
| DE22-007 | — | 1.0E+07 | — | 1.0E+07 | GutPlus ® |
| DE22-008 | — | — | 1.0E+07 | 1.0E+07 | Ecobiol ® |
| DE22-009 | — | — | 1.0E+06 | 1.0E+06 | Ecobiol ® |
| DE22-010 | — | — | — | — | Blank |

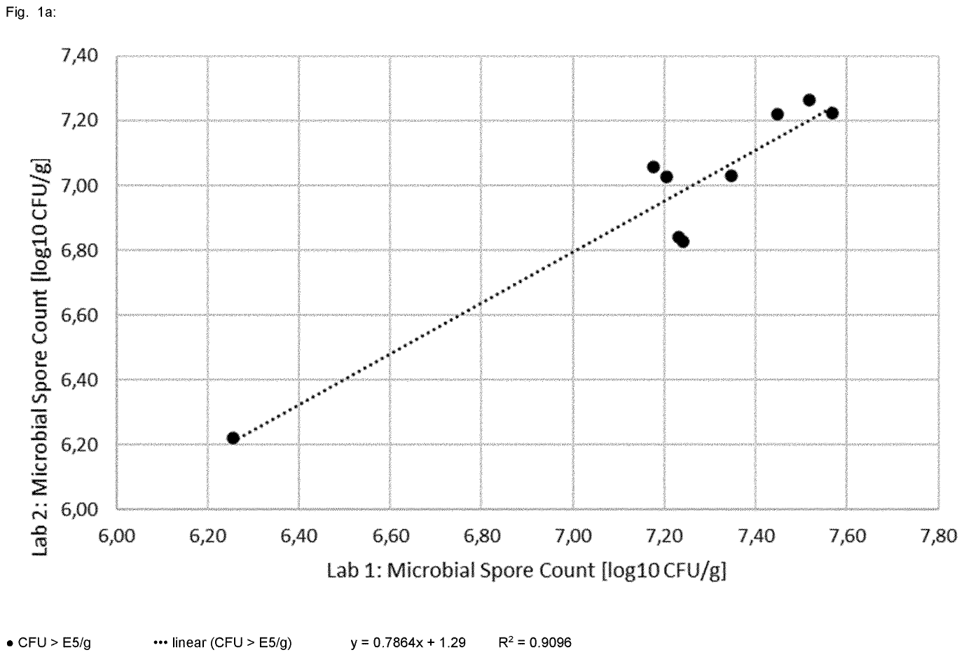

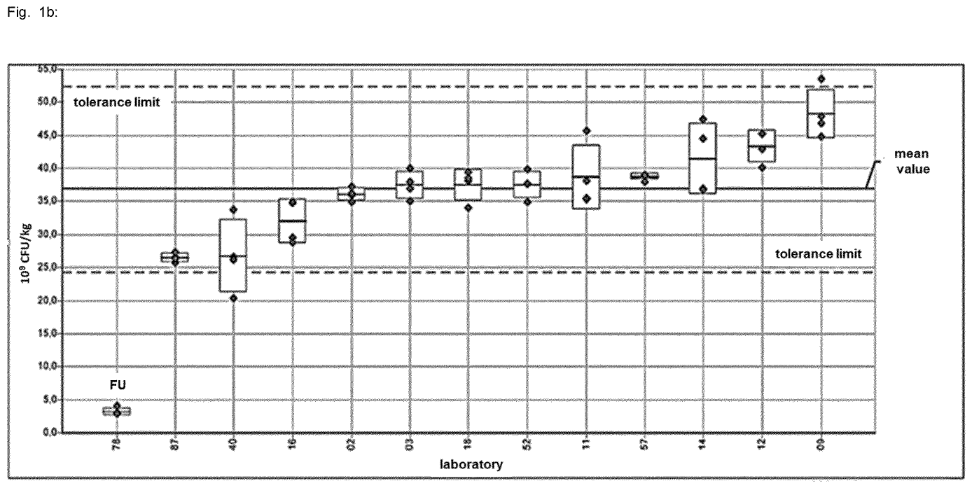

In total 9 different samples with the composition of Table 7 were measured with the method according to the present invention and with the microbial spore count, i.e., the method not according to the present invention. The thus obtained measurement results are shown in the FIGS. 1a) to 14 and these results are discussed in detail below.

FIG. 1a) shows the correlation of microbial spore count between Lab 1 (external laboratory) and Lab 2 (QA-Laboratory Evonik). A linear regression was fitted for the measured samples and determined to the decadic logarithm (Log 10) of colony forming units per gram (CFU/g) and resulted in a good linearity with a coefficient of determination R²>0.90. The equation of the regression curve is f(x)=0.7864*x+1.29.

FIG. 1b) is a box plot representation of the number of colony-forming-units (CFU) within the scope of the interlaboratory test of microbial spore count between the participated laboratories (N=13). The evaluation of the results are reported from VDLUFA (Verband Deutscher Landwirtschaftlicher Untersuchungs- und Forschungsanstalten). Box shows median and interquartile range. Based on this information, the average was 3.70E+07 CFU/g, resulting with a tolerance range between 2.43 E+07 to 5.24 E+07 CFU/g. The inaccuracy of the CFU determination has defined with a relative variance of 18.64%.

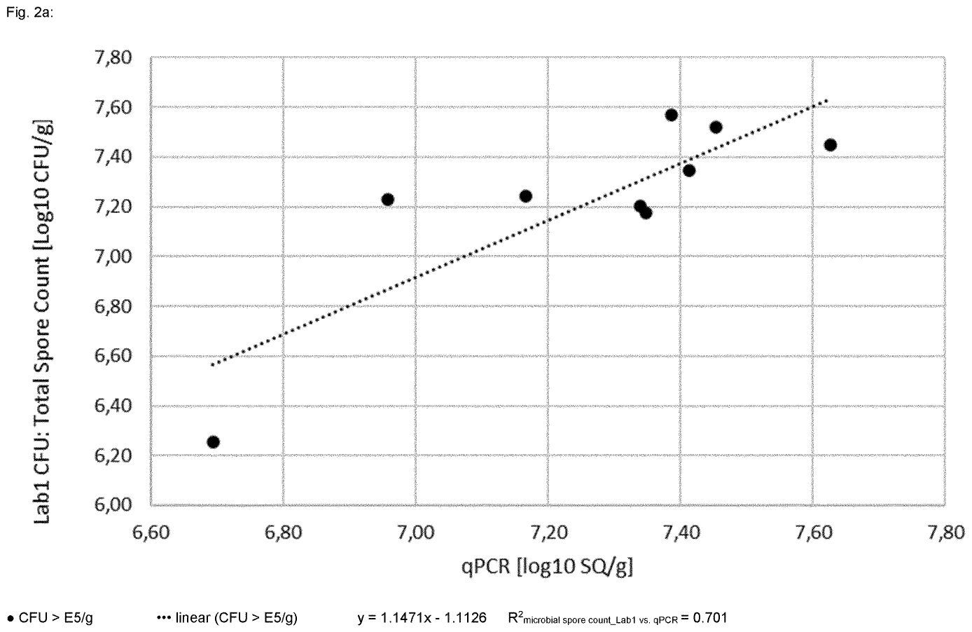

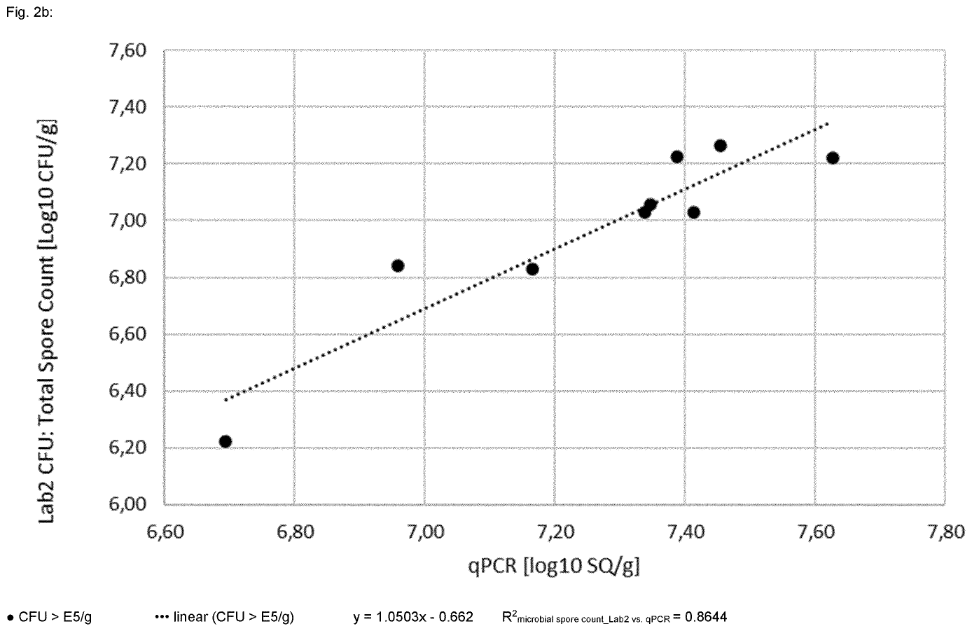

FIGS. 2a) and 2b) illustrate the linear regressions between the values determined with the method according to the quantitative PCR and with the microbial spore count (CFU), in which the measurements were correlated with Lab1 (FIG. 2a) and Lab 2 (FIG. 2b). Both curves can be established by plotting the concentration of starting quantity (SQ) expressed as the decadic logarithm on the x-axis (log scale) against the decadic logarithm of colony forming units per gram (CFU/g). In sum, 9 feed samples from broiler (DE22-001 to DE22-009) with known spore counts ranging from 1.0 E+06 to 2.0 E+07 spores per gram were analyzed and calculated with 3 valid replicates. Linear correlations were calculated for the respective regression curves and indicated a moderate positive linear correlation between the considered laboratories with R²_{microbial spore count_Lab1 vs. qPCR}=0.701 and R²_{Microbial spore count_Lab2 vs. qPCR}=0.864. The difference between the two methods results from the different detection of cell type, quantified by each method: while the microbial spore count only quantifies spores, which are able to germinate under specific growth conditions, the molecular method determines all bacterial cells containing the target ID fragment, independent if they are present as alive, dead, vegetative or germinable cells.

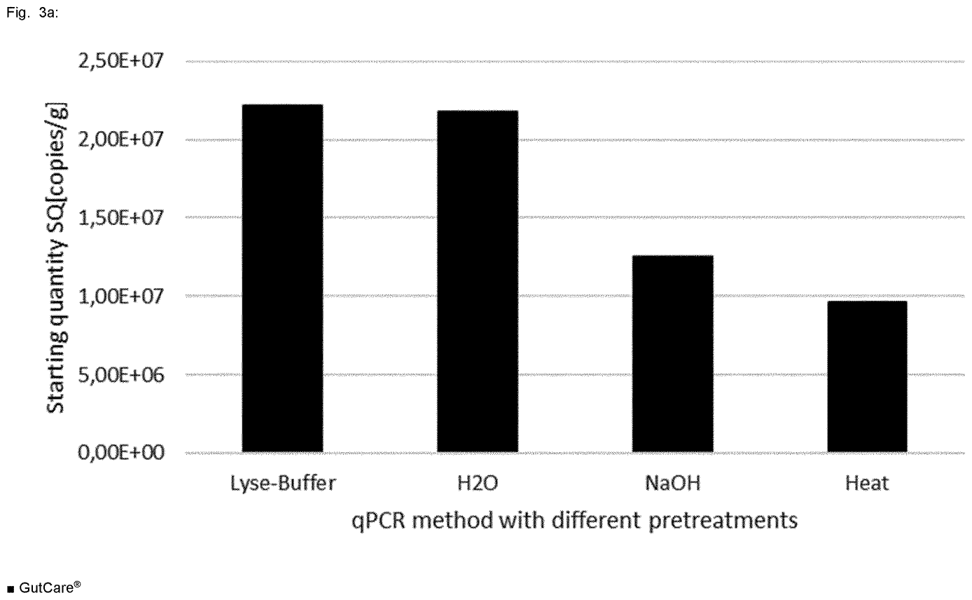

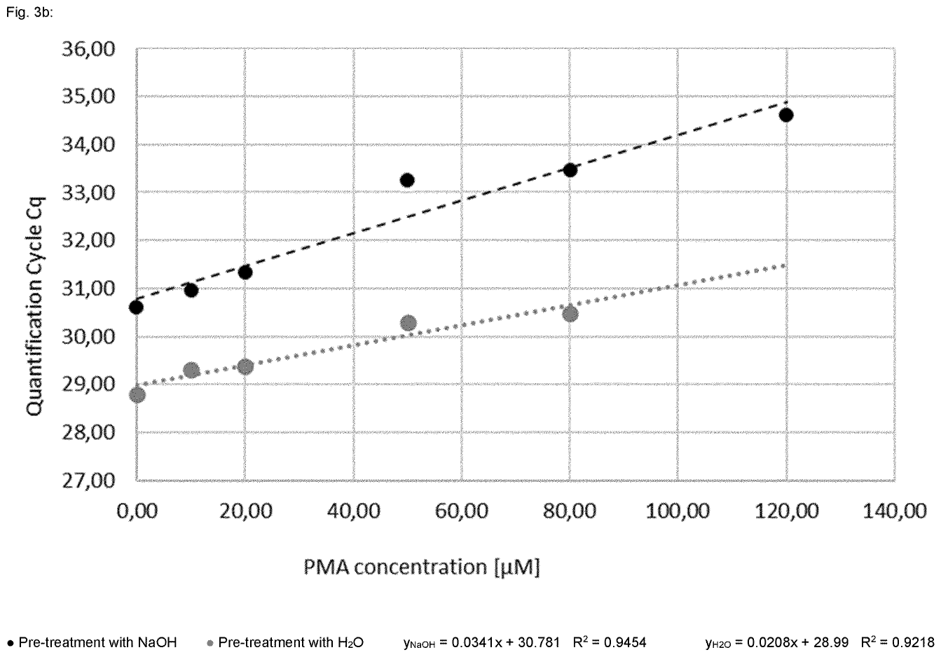

FIG. 3a) shows the evaluation of the different sample pre-treatments without the addition of DNA binding dye propidium monoazide (PMA) for the GutCare® feed sample DE22-006 with the strain B. subtilis DSM 32315. In order to evaluate the feasibility and the impact of different pre-treatments the tested feed sample was subjected to pre-treatment in solutions of lyse-buffer, pure water (H₂O), sodium hydroxide (NaOH) and alternatively to a heat pre-treatment. Both pre-treatments with sodium hydroxide and heat at 80 °C are used in the microbial spore count. The concentrations [SQ/g] to the respective treatment are as follows, pre-treatment with lyse-buffer 2.22 E+07; pre-treatment with pure water 2.18 E+07; pre-treatment with sodium hydroxide 1.25 E+07; pre-treatment with heat 9.65 E+07.

FIG. 3b) displays the linearity of the PMA concentration and covers a range from 120 μM to 0 μM. Both curves can be established by plotting the concentration of PMA in micromolar on the x-axis against the quantification cycle (Cq) on the y-axis. The linearity is carried out for the pre-treatment with sodium hydroxide and the pre-treatment with pure water for the broiler feed sample with GutCare®. In order to measure the germinable cells in a feed sample, sodium hydroxide is used as part of sample pre-treatment to lyse all vegetative cells and therefore affected to quantify only the germinable cells as proceed using the microbial spore count. The linearity curve of sodium hydroxide is shifted upwards along the y-axis compared to the sample pre-treatment with water, which means the number of cycles increase with sodium hydroxide and resulting reduction of cell concentration due to the selection of vegetative cells and non-viable cells. The regression curves show a clear trend towards higher Cq with increasing the PMA concentration. The higher the dye concentration, the more easily PMA can also penetrate intact cells. Both curves are asynchronous at a PMA concentration of 50 μM. An interaction takes place at the higher dye concentration between the suspension with pre-treatment solution and DNA binding dye propidium monoazide. PMA with concentration of 20 μM and 50 μM are used for these measurements.

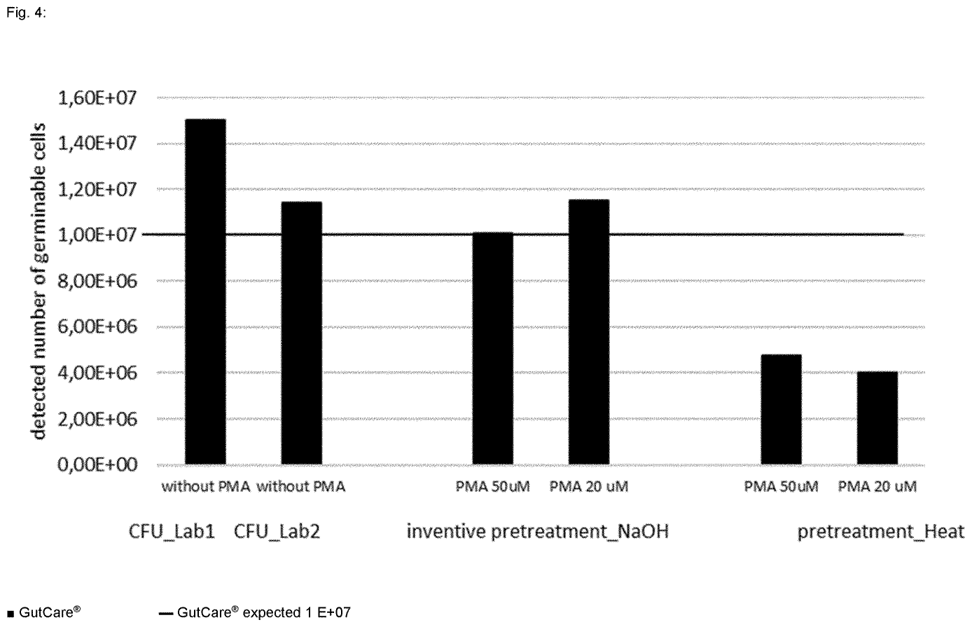

FIG. 4 illustrates the number of germinable cells determined with the method according to the present invention for the strain B. subtilis DSM 32315 with different pre-treatment methods in comparison to the microbial spore count for GutCare® feed sample DE22-006. Pretreatment with sodium hydroxide and the PMA concentration of 20 μM revealed the slight relative deviation between the method according to the present invention and microbial spore count and with PMA concentration of 50 μM to the number of germinable cells approximate the expected concentration of 1E+07 CFU/g. Pretreatment with heat shows the largest deviation, probably due to denaturing effects of heating on the DNA. Following concentrations of germinable cells are resulted from the respective determination method: Microbial spore count Lab1 1.50E+07 CFU/g; Microbial spore count Lab2 1.14E+07 CFU/g; inventive method with NaOH-pretreatment with 50 μM PMA 1.01E+07 SQ/g; inventive method with NaOH-pretreatment with 20 μM PMA 1.15E+07 SQ/g; inventive method with heat-pretreatment with 50 μM PMA 4.75E+06 SQ/g; inventive method with heat-pretreatment with 20 μM PMA 4.03E+06 SQ/g.

Table 8 shows the converted results from FIG. 4 to the relative deviation between Log 10 SQ/g determined with the method according to the present invention and Log 10 CFU/g determined with the microbial spore count by both laboratories. In general, all results of pre-treatments have shown a relative difference to the microbial spore count less than 8%. The method according to the present invention with pre-treatment with sodium hydroxide NaOH shows the smallest deviation to the microbial spore count of laboratory 2 for the PMA concentrations 20 μM and 50 μM.

TABLE 8
Relative deviation between the results obtained with the method according to the present
invention (Log10 SQ/g) and with the microbial spore count (Log10 CFU/g) for both
laboratories (converted results for GutCare ® from FIG. 4).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| GutCare ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −2.39 | −0.74 | −6.96 | −5.39 |
| 20 μM/1.0 μL | −1.59 | 0.08 | −7.96 | −6.40 |

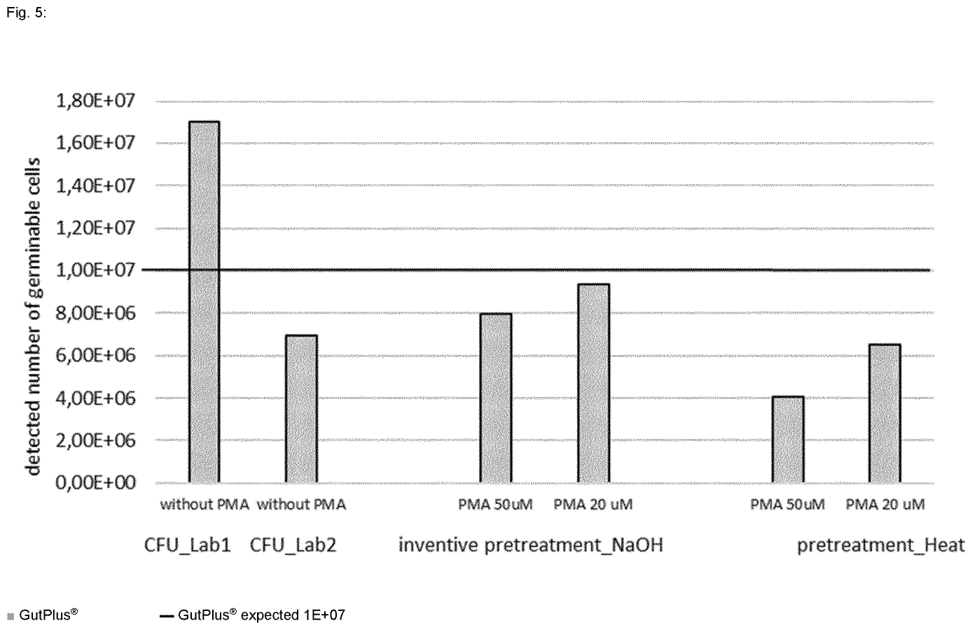

FIG. 5 illustrates the number of germinable cells determined with the method according to the present invention for the strain B. subtilis DSM 32540 with different pre-treatment methods in comparison to the microbial spore count for GutPlus® feed sample DE22-007. Pre-treatment with sodium hydroxide and the PMA concentration of 50 μM revealed the slight relative deviation between the method according to the present invention and the microbial spore count and PMA concentration of 20 μM gets closer to the expected concentration of 1E+07 CFU/g. Pre-treatment with heat shows the largest deviation, probably due to denaturing effects of heating on the DNA. Following concentrations of germinable cells are resulted from the respective determination method: Microbial spore count Lab1 1.70E+07 CFU/g; Microbial spore count Lab2 6.93E+06 CFU/g; inventive method with NaOH-pretreatment with 50 μM PMA 7.97E+06 SQ/g; inventive method with NaOH-pretreatment with 20 μM PMA 9.35E+06 SQ/g; inventive method with heat-pretreatment with 50 μM PMA 4.04E+06 SQ/g; inventive method with heat-pretreatment with 20 μM PMA 6.51E+06 SQ/g.

Table 9 shows the converted results from FIG. 5 to the relative deviation between Log 10 SQ/g obtained with the method according to the present invention and Log 10 CFU/g for the microbial spore count determined by both laboratories. In general, all results of pre-treatments have shown a relative difference to the microbial spore count less than 9%. The method according to the present invention with pre-treatment with sodium hydroxide NaOH shows the smallest deviation to the microbial spore count for laboratory 2 for the PMA concentrations 20 μM and 50 μM.

TABLE 9
Relative deviation between the results obtained with the method according to the present
invention (Log10 SQ/g) and with the microbial spore count (Log10 CFU/g) for both
laboratories (converted results for GutPlus ® from FIG. 5).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| GutPlus ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −4.55 | 0.88 | −8.63 | −3.43 |
| 20 μM/1.0 μL | −3.59 | 1.90 | −5.77 | −0.40 |

FIG. 6 illustrates the number of germinable cells determined with the method according to the present invention for the strain B. amyloliquefaciens CECT 5940 with different pre-treatment methods in comparison to the microbial spore count for Ecobiol® feed sample DE22-008 determined by both laboratories. Pretreatment with sodium hydroxide and the PMA concentration of 50 μM revealed the slight relative deviation between the results determined with the method according to the present invention and with the microbial spore count and gets closer to the expected concentration of 1E+07 CFU/g. Pretreatment with heat shows the largest deviation, probably due to denaturing effects of heating on the DNA. Following concentrations of germinable cells are resulted from the respective determination method: Microbial spore count_Lab1 1.60E+07 CFU/g; Microbial spore count Lab2 1.07E+07 CFU/g; inventive method with NaOH-pretreatment with 50 μM PMA 9.91E+06 SQ/g; inventive method with NaOH-pretreatment with 20 μM PMA 1.39E+07 SQ/g; inventive method with heat-pretreatment with 50 μM PMA 5.96E+06 SQ/g; inventive method with heat-pretreatment with 20 μM PMA 7.07E+06 SQ/g.

Table 10 shows the converted results from FIG. 6 to the relative deviation between Log 10 SQ/g determined with the method according to the present invention and Log 10 CFU/g with the microbial spore count by both laboratories. In general, all results of pre-treatments have shown a relative difference to the microbial spore count less than 6%. The method according to the present invention with pre-treatment with sodium hydroxide NaOH shows the smallest deviation to the microbial spore count of for laboratory 2 for the PMA concentrations 20 μM and 50 μM.

TABLE 10
Relative deviation between the results obtained with the method according to the present
invention (Log10 SQ/g) and with the microbial spore count (Log10 CFU/g) for both
laboratories (converted results for Ecobiol ® from FIG. 6).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| Ecobiol ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −2.89 | −0.48 | −5.95 | −3.61 |
| 20 μM/1.0 μL | −0.85 | 1.62 | −4.93 | −2.56 |

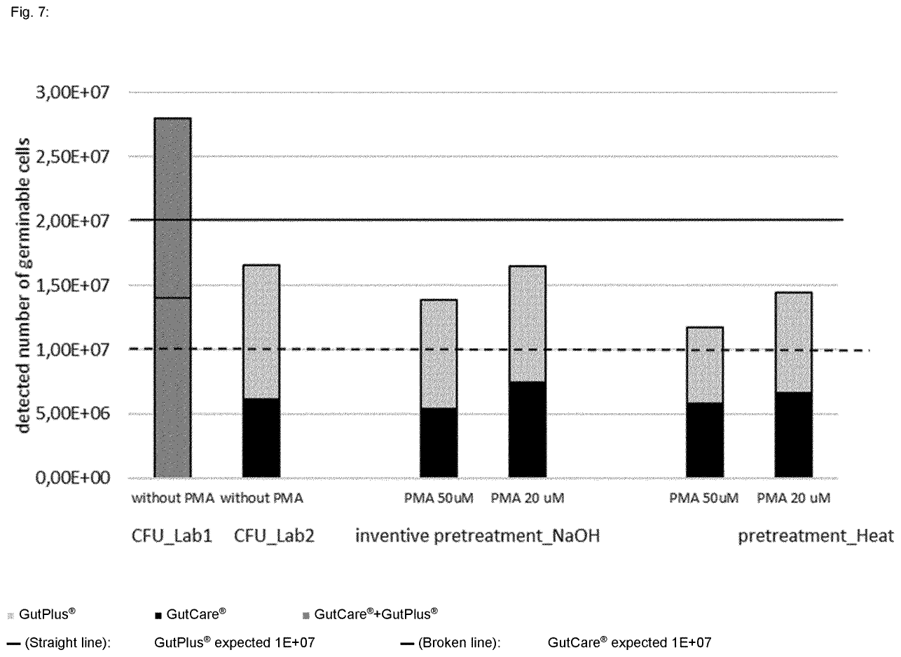

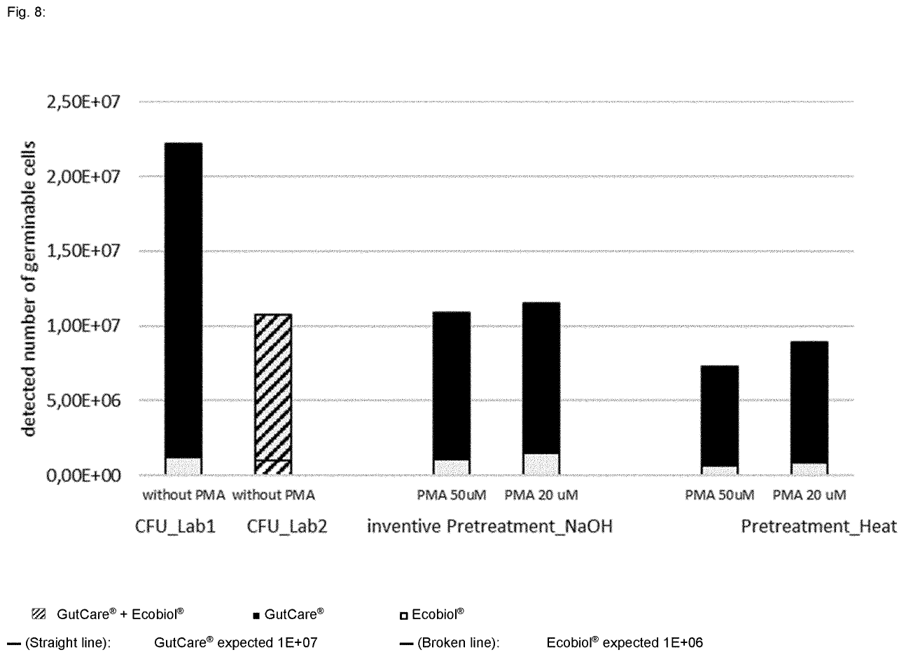

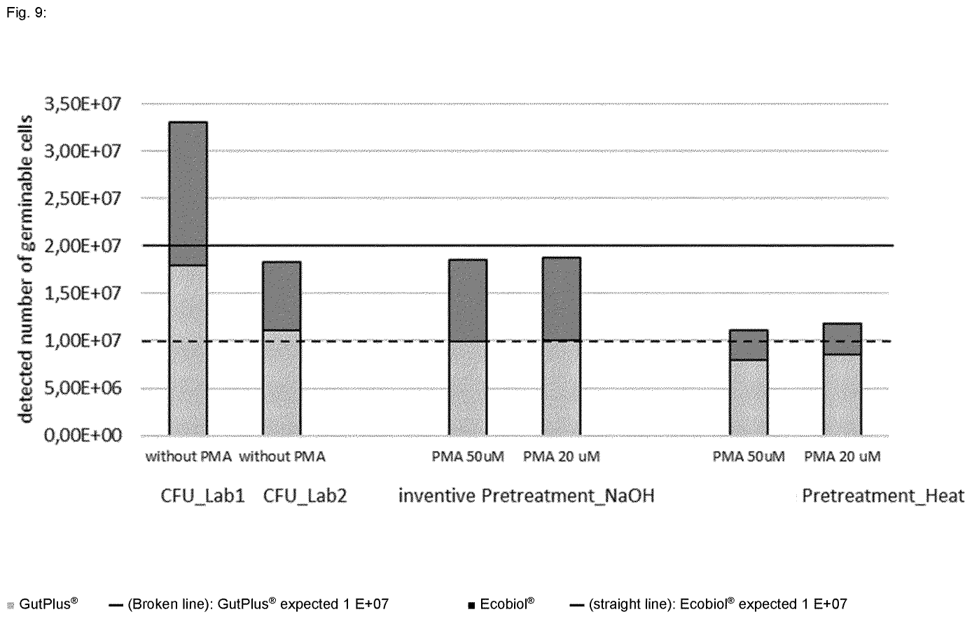

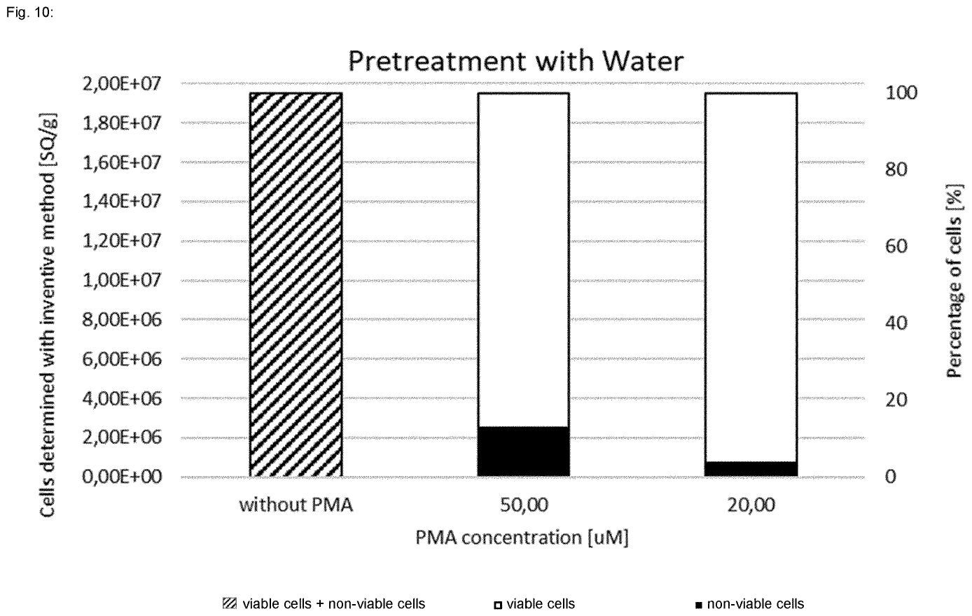

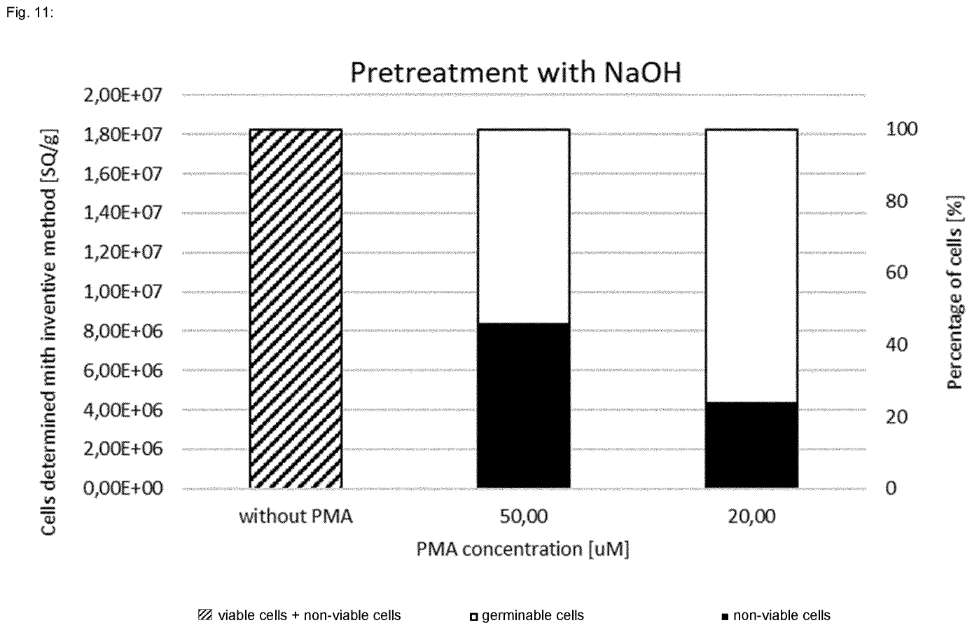

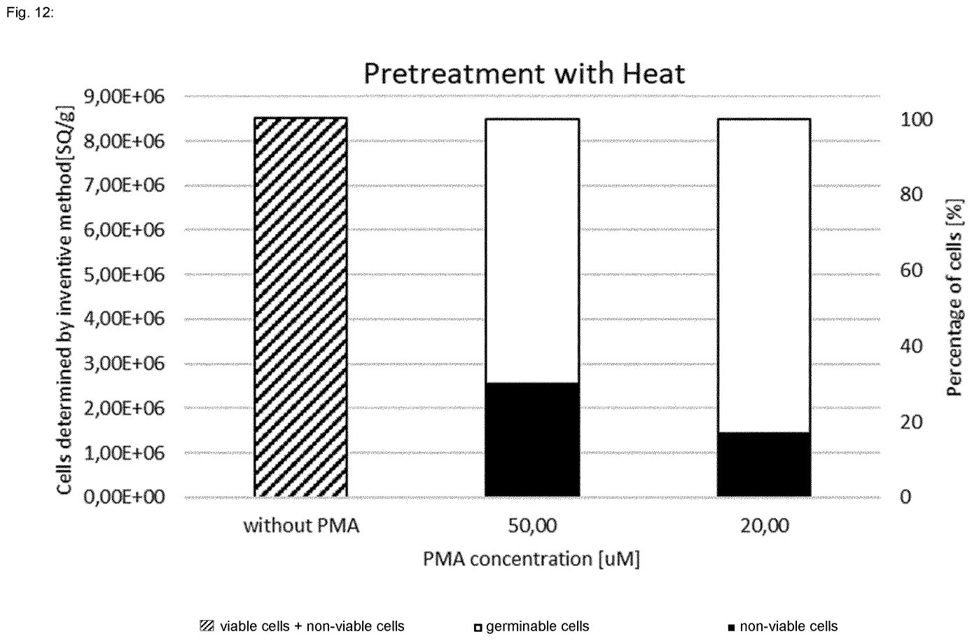

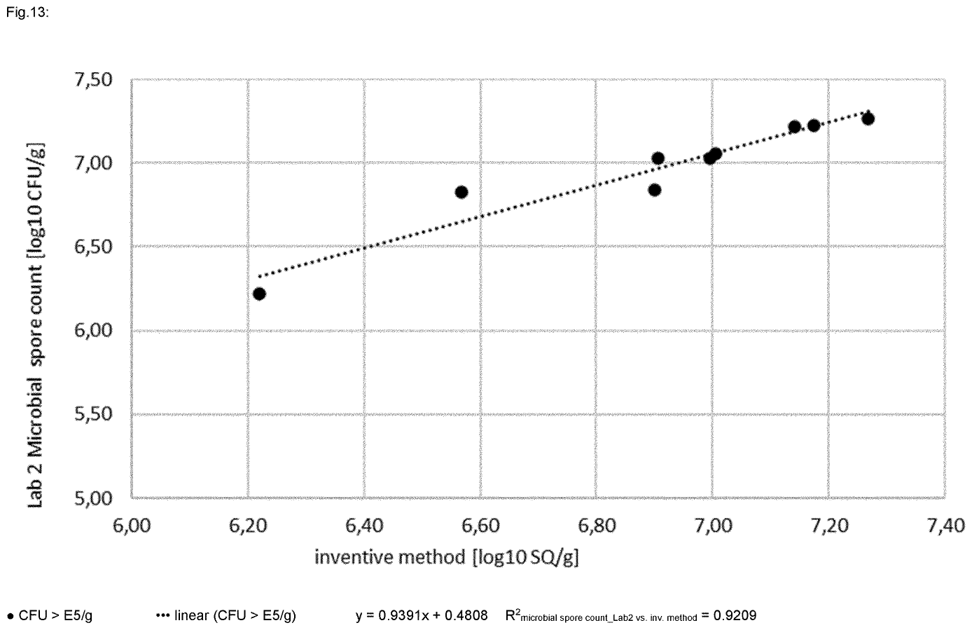

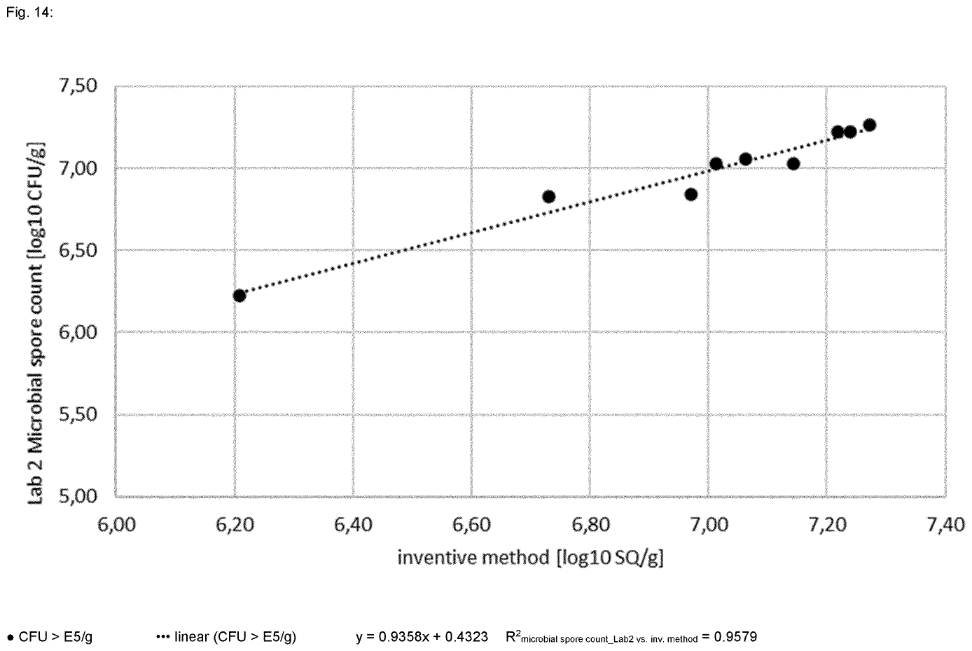

FIG. 7 illustrates the number of germinable cells determined with the method according to the present invention for the combination strain of B. subtilis DSM 32315 (GutCare®) and B. subtilis DSM 32540 (GutPlus®) with different pre-treatment methods in comparison to the microbial spore count for DE22-001 feed sample determined by both laboratories.

Laboratory 1 (Lab1) was unable to differentiate both strains so that only the microbial spore count as sum of both products is indicated in FIG. 7. The following concentrations of germinable cells for the respective strain and determination method were determined: Microbial spore count Lab1 (microbial spore count as sum of both products) 2.80E+07 CFU/g; Microbial spore count for GutCare® 6.17E+06 CFU/g and GutPlus® 1.04E+07 CFU/g; inventive method with NaOH-pretreatment with 50 μM PMA for GutCare® 5.43E+06 SQ/g and GutPlus® 8.38E+06 SQ/g; inventive method with NaOH-pretreatment with 20 μM PMA for GutCare® 7.45E+06 SQ/g and GutPlus® 9.07E+06 SQ/g; inventive method with heat-pretreatment with 50 μM PMA for GutCare® 5.81E+06 SQ/g and GutPlus® 5.90E+06 SQ/g and; inventive method with heat-pretreatment with 20 μM PMA for GutCare® 6.63E+06 SQ/g and GutPlus® 7.82E+06 SQ/g.

Tables 11, 12 and 13 show the converted results from FIG. 7 to the relative deviation for the respective target strains between Log 10 SQ/g determined with the method according to the present invention and Log 10 CFU/g for the microbial spore count determined by both laboratories. In general, all results of pre-treatments have shown a relative difference to the microbial spore count in Table 11 less than 1.3%, in Table 12 less than 3.5% and in Table 13 less than 4.1%. The method according to the present invention with pre-treatment with sodium hydroxide NaOH shows the smallest deviation to the microbial spore count of laboratory 2 for the PMA concentrations 20 μM and 50 μM.

TABLE 11
Relative deviation between the results obtained with the method according to the present
invention (Log10 SQ/g) and with the microbial spore count (Log10 CFU/g) for both
laboratories (converted results for GutCare ® from FIG. 7).

| | Deviation | Deviation | Deviation | Deviation |
|---|---|---|---|---|
| | between | between | between | between |
| | inventive method | inventive method | inventive method | inventive method |
| | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | n.a. | −0.81 | n.a. | −0.39 |
| 20 μM/1.0 μL | n.a. | 1.21 | n.a. | 0.46 |

TABLE 12
Relative deviation between the results obtained with the method according to the present
invention (Log10 SQ/g) and with the microbial spore count (Log10 CFU/g) for both
laboratories (converted results for GutPlus ® from FIG. 7).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| GutPlus ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | n.a. | −1.33 | n.a. | −3.51 |
| 20 μM/1.0 μL | n.a. | −0.85 | n.a. | −1.76 |

TABLE 13
Relative deviation between the results obtained with the method according
to the present invention (Log10 SQ/g) and with the microbial
spore count (Log10 CFU/g) for both laboratories (converted
results for the combination of GutCare ® and GutPlus ® from FIG. 7).

| | Pre-treatment with NaOH | | Pre-treatment with heat | |
|---|---|---|---|---|
| | Deviation | Deviation | Deviation | Deviation |
| | between | between | between | between |
| GutCare ® + | inventive method | inventive method | inventive method | inventive method |
| GutPlus ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −4.12 | −1.09 | −5.09 | −2.09 |
| 20 μM/1.0 μL | 3.08 | −0.02 | −3.86 | −0.82 |

FIG. 8 illustrates the number of germinable bacteria cells for the combination strain of B. subtilis DSM 32315 (GutCare®) and B. amyloliquefaciens CECT 5940 (Ecobiol®) determined with the method according to the present invention with different pre-treatment methods in comparison to the microbial spore count for DE22-004 feed sample determined by both laboratories.

Laboratory 2 (Lab2) was unable to differentiate both strains from each other so that only the microbial spore count as sum of both products is indicated in FIG. 8. Following concentrations of germinable cells for the respective strain and determination method were determined: Microbial spore count Lab1 (microbial spore count as sum of both products) 1.07E+07 CFU/g; Microbial spore count Lab1 for Ecobiol® 1.20E+06 CFU/g and GutCare® 2.10E+07 CFU/g; inventive method with NaOH-pretreatment with 50 μM PMA for Ecobiol® 1.09E+06 SQ/g and GutCare® 9.82E+06 SQ/g; inventive method with NaOH-pretreatment with 20 μM PMA for Ecobiol® 1.50E+06 SQ/g and GutCare® 1.01E+07; inventive method with heat-pretreatment with 50 μM PMA for Ecobiol® 6.13E+05 and GutCare® 6.67E+06 SQ/g and; inventive method with heat-pretreatment with 20 μM PMA for Ecobiol® 8.65E+05 and GutCare® 8.02E+06 SQ/g.

Tables 14, 15 and 16 show the converted results from FIG. 8 to the relative deviation for the respective target strains between Log 10 SQ/g determined with the method according to the present invention and Log 10 CFU/g for the microbial spore count determined by both laboratories. In general, all results of pre-treatments have shown a relative difference to the microbial spore count in Table 14 less than 6.8%, in Table 15 less than 4.8% and in table 16 less than 6.6%. The method according to the present invention with pre-treatment with sodium hydroxide NaOH shows the smallest deviation to the microbial spore count of laboratory 2 for the PMA concentrations 20 μM and 50 μM.

TABLE 14
Relative deviation between the results obtained with the method according to the present
invention (Log10 SQ/g) and with the microbial spore count (Log10 CFU/g) for both
laboratories (converted results for GutCare ® from FIG. 8).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| GutCare ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −4.51 | n.a. | −6.81 | n.a. |
| 20 μM/1.0 μL | −4.37 | n.a. | −5.71 | n.a. |

TABLE 15
Relative deviation between the results obtained with the method according to the present
invention (Log10 SQ/g) and with the microbial spore count (Log10 CFU/g) for both
laboratories (converted results for Ecobiol ® from FIG. 8).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| Ecobiol ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −0.70 | n.a. | −4.80 | n.a. |
| 20 μM/1.0 μL | 1.61 | n.a. | −2.34 | n.a. |

TABLE 16
Relative deviation between the results obtained with the method according
to the present invention (Log10 SQ/g) and with the microbial spore
count (Log10 CFU/g) for both laboratories (converted
results for the combination of GutCare ® and Ecobiol ® from FIG. 8).

| | Pre-treatment with NaOH | | Pre-treatment with heat | |
|---|---|---|---|---|
| | Deviation | Deviation | Deviation | Deviation |
| | between | between | between | between |
| GutCare ® + | inventive method | inventive method | inventive method | inventive method |
| Ecobiol ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −4.20 | 0.10 | −6.59 | −2.39 |
| 20 μM/1.0 μL | −3.86 | 0.46 | −5.42 | −1.16 |

FIG. 9 illustrates the number of germinable bacteria cells for the combination strain of B. subtilis DSM 32540 (GutPlus®) and B. amyloliquefaciens CECT 5940 (Ecobiol®) determined with the method according to the present invention with different pre-treatment methods in comparison to the microbial spore count for the DE22-003 feed sample.

Both laboratories determining the microbial spore count were able to differentiate both strains. The following concentrations were determined for germinable cells of the respective strain with different methods: Microbial spore count Lab1 for GutPlus® 1.80E+07 CFU/g and Ecobiol® 1.50E+07 CFU/g; microbial spore count Lab2 for GutPlus® 1.11 E+07 CFU/g and Ecobiol® 7.28E+06 SQ/g CFU/g; inventive method with NaOH-pretreatment with 50 μM PMA for GutPlus® 9.92E+06 SQ/g and Ecobiol® 8.62E+06 SQ/g; inventive method with NaOH-pretreatment with 20 μM PMA for GutPlus® 1.00E+07 and Ecobiol® 8.67E+06 SQ/g; inventive method with heat-pretreatment with 50 μM PMA for GutPlus® 8.01E+06 SQ/g and Ecobiol® 3.15E+06 SQ/g and; inventive method with heat-pretreatment with 20 μM PMA for GutPlus® 8.55E+06 SQ/g and Ecobiol® 3.20E+06 SQ/g.

Tables 17, 18 and 19 shows the converted results from FIG. 9 to the relative deviation for the respective target strains between Log 10 SQ/g determined with the method according to the present invention and Log 10 CFU/g determined with the microbial spore count by both laboratories. In general, all results of pre-treatments have shown a relative difference to the microbial spore count in Table 17 less than 5.0%, in table 18 less than 9.5% and in Table 19 less than 6.3%. The method according to the present invention with pre-treatment with sodium hydroxide NaOH shows the smallest deviation to the microbial spore count of laboratory 2 for the PMA concentrations 20 μM and 50 μM.

TABLE 17
Relative deviation between the results obtained with method according to the present
invention (Log10 SQ/g) and with the microbial spore count (Log10 CFU/g) for both
laboratories (converted results for GutPlus ® from FIG. 9).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| GutPlus ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −3.56 | −0.67 | −4.84 | −1.99 |
| 20 μM/1.0 μL | −3.50 | −0.60 | −4.46 | −1.59 |

TABLE 18
Relative deviation between the results obtained with the method according to the present
invention (Log10 SQ/g) and with the microbial spore count (Log10 CFU/g) for both
laboratories (converted results for Ecobiol ® from FIG. 9).

| | Pre-treatment with NaOH | | Pre-treatment with heat | |
|---|---|---|---|---|
| | Deviation | Deviation | Deviation | Deviation |
| | between | between | between | between |
| | inventive method | inventive method | inventive method | inventive method |
| Ecobiol ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −0.70 | n.a. | −4.80 | n.a. |
| 20 μM/1.0 μL | 1.61 | n.a. | −2.34 | n.a. |

TABLE 19
Relative deviation between the results obtained with the method according to the present
invention (Log10 SQ/g) and with the microbial spore count (Log10 CFU/g) for both
laboratories (converted results for the combination of GutPlus ®
and Ecobiol ® from FIG. 9).

| | Pre-treatment with NaOH | | Pre-treatment with heat | |
|---|---|---|---|---|
| | Deviation | Deviation | Deviation | Deviation |
| | between | between | between | between |
| GutPlus ® + | inventive method | inventive method | inventive method | inventive method |
| Ecobiol ® | and microbial | and microbial | and microbial | and microbial |
| PMA conc. [μM]/ | spore count | spore count | spore count | spore count |
| PMA vol. [μL] | Lab1 | Lab2 | Lab1 | Lab2 |
| 50 μM/2.5 μL | −0.70 | n.a. | −4.80 | n.a. |
| 20 μM/1.0 μL | 1.61 | n.a. | −2.34 | n.a. |

FIGS. 10, 11 and 12 represent the measured Ecobiol® feed sample DE22-008 with the single strain B. amyloliquefaciens CECT 5940 and the resulting definition of the bacterial cell structure groups in respect of the three pre-treatments for the PMA concentrations of 20 μM and 50 μM.

FIG. 10 shows the results that were measured with the method according to the present invention with the pre-treatment involving pure water. This bar chart refers the total concentration, which presents the viable and non-viable cells and gives a differentiated consideration of viable (germinable and vegetative) and non-viable cells, expressed as a SQ/g. Without the addition of PMA, 1.95E+07 SQ/g were determined, including viable and non-viable cells. The addition of PMA aims to distinguish between the viable and non-viable cells. With PMA concentration of 50 μM a number of viable cells of 1.70E+07 SQ/g and non-viable cells of 2.49E+06 SQ/g were determined and in case of 20 μM to a number of viable cells of 1.88E+07 SQ/g and non-viable cells 7.04E+05 SQ/g respectively.

FIG. 11 shows the results for the sample pre-treatment with sodium hydroxide NaOH and FIG. 12 shows the sample pre-treatment with heat, which were measured with the method according to the present invention with the respective pre-treatments. This bar chart refers the total concentration, which presents the viable and non-viable cells and gives a differentiated consideration of the group of viable cells including germinable and vegetative cells. Both pre-treatment methods aim to lyse the vegetative cells in a sample to ensure the qualitative and quantitative determination of germinable cells. All results are expressed as a SQ/g.

In FIG. 11 the obtained concentration without PMA were accounted with 1.83E+07 SQ/g, which includes all viable and non-viable cells. Addition of PMA of 50 μM led to a concentration of 9.91E+06 SQ/g germinable cells and PMA of 20 μM 7.07E+06 SQ/g respectively.

In FIG. 12 the obtained concentration without PMA were accounted with 8.52E+06 SQ/g. With PMA of 50 μM 5.96E+06 SQ/g germinable cells were determined and with PMA of 20 μM 7.07E+06 SQ/g germinable cells were determined.

FIG. 13 (application of 50 μM PMA) and FIG. 14 (application of with 20 μM PMA) with the pre-treatment of sodium hydroxide illustrate the linear regressions between the values obtained with the method according to the present invention and with the microbial spore count, determined by laboratory 2. Both curves can be established by plotting the concentration of starting quantity (SQ) expressed as the decadic logarithm on the x-axis (log scale) against the decadic logarithm of colony forming units per gram (CFU/g). In sum, 9 feed samples from broiler with known microbial spore counts ranging from 1.0E+06 to 2.0E+07 CFU/g were analyzed and calculated with 3 valid replicates. Linear correlations were calculated for the respective regression curves and indicated an excellent linear correlation between the considered methods with R²_{microbial spore count_Lab2 vs. inv. method_PMA50 μM}=0.921 and R²_{microbial spore count_Lab2 vs. inv. method_PMA20 μM}=0.958. The representation of the values determined with the method according to the present invention with the values determined obtained with microbial spore count shows an excellent linear correlation in comparison to the correlations in FIGS. 1a, 2a and 2b. These figures display the consistency of the method according to the present invention with the microbial spore count, which is able to determine only the germinable cells, dependent if the gene originated from a viable or dead cell. However, in comparison to the microbial spore count, the method according to the present invention has robustness and allows for simple manual and rapid determination and sensitivity.

1. A method for determining the number and/or concentration of germinable cells in a sample of biological origin, wherein the germinable cells are bacterial spores formed within vegetative cells, the method comprising:
a) subjecting at least a part of a sample material of biological origin to a pre-treatment comprising a lysis of vegetative cells,
b) photolyzing the pre-treated sample material obtained from a),
c) separating nucleic acids from the photolyzed sample material obtained from b), and
d) subjecting the separated nucleic acids obtained from c) to a quantitative polymerase chain reaction to obtain the number and/or concentration of germinable cells in the sample material of a),
wherein the pre-treatment of a) comprises treating the sample material with a basic solution.

2: The method according to claim 1, wherein a) further comprises subjecting the sample material to an elevated temperature and/or additional mechanic treatment.

3: The method according to claim 1, wherein the base in the basic solution has a pKb of 0 or less.

4: The method according to claim 2, wherein the elevated temperature is a temperature of at least 80 °C.

5: The method according to claim 1, wherein b) comprises
b1) protecting the pre-treated sample material obtained from a) from light,
b2) adding a photoreactive DNA-binding dye to the sample material of b1), and
b3) photolyzing the sample material obtained from b2).

6: The method according to claim 5, wherein the photoreactive DNA-binding dye intercalates or penetrates into DNA of cells with a disturbed wall structure and/or cells with a damaged cell wall structure comprising a cortex region and an inner membrane wall.

7: The method according to claim 5, wherein the photoreactive DNA-binding dye is propidium monoazide, ethidium bromide monoazide, a derivative thereof, or a mixture thereof.

8: The method according to claim 1, wherein c) comprises
c1) subjecting the photolyzed sample material obtained from b) to a chemical and/or mechanical lysis to release nucleic acids from the sample material,
c2) binding the released nucleic acids obtained from c1) to beads to obtain loaded beads,
c3) washing the loaded beads obtained from c2), and
c4) releasing the nucleic acids from the washed beads obtained from c3) by treatment with an elution buffer or water to obtain a solution comprising the released nucleic acids.

9: The method according to claim 8, wherein the chemical lysis in c1) is performed under chaotropic conditions.

10: The method according to claim 8, wherein the chemical lysis in c1) is performed under chaotropic high salt conditions.

11: The method according to claim 1, further comprising
e) determining the number and/or concentration of non-viable and vegetative cells in the sample of a), comprising
e1) subjecting a part of the sample material of a) to a determination of the number and/or concentration of all cells in the sample material, and
e2) taking a difference between the number and/or concentration of all cells obtained in e1) and the number and/or concentration of germinable cells obtained in e) to obtain the number and/or concentration of the non-viable cells and the vegetative cells in the sample material of a).

12: The method according to claim 1, further comprising
f) determining the number and/or concentration of the vegetative cells in the sample of a), comprising
f1) subjecting a part of the sample material of a) to a determination of the number and/or concentration of the viable cells in said sample material, and
f2) taking a difference between the number and/or concentration of the viable cells obtained in f1) and the number and/or concentration of the germinable cells obtained in d) to obtain the number and/or concentration of the vegetative cells in the sample material of a).

13: The method according to claim 1, further comprising
g) determining a relative change in the number and/or concentration of germinable cells, relative to the number and/or concentration of all cells, according to the following formula
$$rel . ( \% ) = \frac{{n}_{g} - {n}_{all}}{{n}_{all}} \times 100 and / or$$
$$rel . ( \% ) = \frac{{c}_{g} - {c}_{all}}{{c}_{all}} \times 100$$
wherein
rel. (%) is the relative change in the number and/or concentration of germinable cells, in percent,
n_{g} is the number of germinable cells, obtained in d),
c_{g} is the concentration of germinable cells, obtained in step d)
nₐₗₗ is the number of all cells, obtained in e1), and
cₐₗₗ is the concentration of all cells, obtained in e1).

14: The method according to claim 1, wherein the sample of a) is a biomass, feed, fecal, or digesta sample.
